SEQUENCE LISTING (11 sequences)

SEQ ID NO: 1 - protein, 217 aa, Artificial Sequence
  Fcab-1 (modified FS1-60
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELDEGGPVSLTCLVKGFYPSDIAVEWESTYGPENNYKTTPPVLDSDGSFFLYSRLTVSHWRWYSGNVFSCSVMHEALHNHYTQKSLSLSPG

SEQ ID NO: 2 - protein, 217 aa, Artificial Sequence
  Fcab-2 (modified FS1-65)
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELDEGGPVSLTCLVKGFYPSDIAVEWESTYGPENNYKTTPPVLDSDGSFFLYSKLTVSYWRWVKGNVFSCSVMHEALHNHYTQKSLSLSPG

SEQ ID NO: 3 - protein, 216 aa, Artificial Sequence
  Fcab-3 (modified FS1-67)
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDETDDGPVSLTCLVKGFYPSDIAVEWESTYGPENNYKTTPPVLDSDGSFFLYSKLTVSYWRWYKGNVFSCSVMHEALHNHYTQKSLSLSPG

SEQ ID NO: 4 - protein, 217 aa, Artificial Sequence
  Fcab-4 (FS1-60[1])
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELDEGGPVSLTCLVKGFYPSDIAVEWESTYGPENNYKTTPPVLDSDGSFFLYSRLTVSHWRWYSGNVFSCSVMHEALHNHYTQKSLSLSPG

SEQ ID NO: 5 - protein, 217 aa, Artificial Sequence
  Fcab-5 (FS1-65)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELDEGGPVSLTCLVKGFYPSDIAVEWESTYGPENNYKTTPPVLDSDGSFFLYSKLTVSYWRWVKGNVFSCSVMHEALHNHYTQKSLSLSPG

SEQ ID NO: 6 - protein, 216 aa, Artificial Sequence
  Fcab-6 (FS1-67)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDETDDGPVSLTCLVKGFYPSDIAVEWESTYGPENNYKTTPPVLDSDGSFFLYSKLTVSYWRWYKGNVFSCSVMHEALHNHYTQKSLSLSPG

SEQ ID NO: 7 - protein, 222 aa, Artificial Sequence
  huFc (modified human IgG1 Fc fragment)
TCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 235 aa, Artificial Sequence
  C-IgG Light chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSLPETGS

SEQ ID NO: 9 - protein, 449 aa, Artificial Sequence
  C-IgG Heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 235 aa, Artificial Sequence; identical to SEQ ID NO: 8
  C-Fab Light chain

SEQ ID NO: 11 - protein, 238 aa, Artificial Sequence
  C-Fab Heavy chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSHHHHHH

(19) United States
(12) Patent Application Publication
(10) Pub. No.: US 2024/0226319 A1
(43) Pub. Date: Jul. 11, 2024

(54) EGFR TARGETING FC ANTIGEN BINDING FRAGMENT-DRUG CONJUGATES

(71) Applicant: MERCK PATENT GMBH, DARMSTADT (DE)

(72) Inventors: Sebastian JAEGER, Darmstadt (DE); Christian SCHROETER, Darmstadt (DE)

(73) Assignee: MERCK PATENT GMBH, DARMSTADT (DE)

(21) Appl. No.: 18/561,506

(22) PCT Filed: May 23, 2022
(86) PCT No.: PCT/EP2022/063839

§ 371 Date: Nov. 16, 2023

(30) Foreign Application Priority Data
May 25, 2021 (EP) 21175808.1

(51) Int. Cl. A61K 47/68; A61K 49/00; A61P 35/00

(52) U.S. Cl. CPC A61K 47/6849; A61K 47/68031; A61K 49/0002; A61P 35/00

(57) ABSTRACT

The invention relates to EGFR targeting Fc antigen binding fragment-drug conjugates (EGFR Fcab-drug conjugates) and the use of the EGFR Fcab-drug conjugates of the present invention for the treatment and/or prevention of hyperproliferative diseases and disorders in mammals, especially humans, and pharmaceutical compositions containing such EGFR Fcab-drug conjugates. Further, the invention relates to EGFR Fcab-label conjugates and diagnostic compositions containing such EGFR Fcab-label conjugates.

The invention relates to EGFR targeting Fc antigen binding fragment-drug conjugates (EGFR Fcab-drug conjugates) and the use of the EGFR Fcab-drug conjugates of the present invention for the treatment and/or prevention of hyperproliferative diseases and disorders in mammals, especially humans, and pharmaceutical compositions containing such EGFR Fcab-drug conjugates. Further, the invention relates to EGFR Fcab-label conjugates and diagnostic compositions containing such EGFR Fcab-label conjugates.

### BACKGROUND OF THE INVENTION

Epidermal growth factor receptor (EGFR; also referred to as ErbB-1 and HER1) is the cell surface receptor for members of the epidermal growth factor family (EGF-family) of extracellular protein ligands. EGFR is a large, monomeric glycoprotein with a single transmembrane region and a cytoplasmic tyrosine kinase domain flanked by noncatalytic regulatory regions. Sequence analyses have shown that the ectodomain contains four subdomains, termed L1, CR1, L2 and CR2, where L and CR are acronyms for large and Cys-rich respectively. The L1 and L2 domains have also been referred to as domains I and III, respectively. The CR domains have been previously referred to as domains II and IV, or as S1.1-S1.3 and S2.1-S2.3 where S is an abbreviation for small.

Cancers which are known to express EGFR include lung cancer (for example non-small cell lung cancer [NSCLC]) (Pao et al., 2010; Amman et al., 2005), glioblastoma multiforme (Taylor et al., 2002), skin cancer (for example cutaneous squamous cell carcinoma) (Uribe et al., 2011), head and neck cancer (such as head and neck squamous-cell carcinoma [HNSCC]) (Zimmermann et al., 2006; Smilek et al., 2012), breast cancer (Masuda et al., 2013), stomach cancer (gastric cancer) (Terashima et al., 2012), colorectal cancer (CRC) (Spano et al., 2005; Saletti et al., 2015), ovarian cancer (Hudson et al., 2009), pancreatic cancer (Troiani et al., 2012), or endometrial cancer (Scambia et al., 1994).

Monoclonal antibodies to the extra-cellular domain of EGFR have been described. These antibodies disrupt ligand binding to EGFR and subsequent signal transduction.

mAbC225 (ERBITUX/cetuximab) is a chimeric IgG1 antibody which binds to the extracellular domain of EGFR and competes with EGF for binding to EGFR, thereby inhibiting downstream pathway signaling and blocking proliferation of tumour cells (Voigt et al., 2012). Cetuximab is FDA approved for the treatment of head and neck cancer, specifically locally or regionally advanced squamous cell carcinoma of the head and neck in combination with radiation therapy, recurrent locoregional disease or metastatic squamous cell carcinoma of the head and neck in combination with platinum-based therapy with 5-FU, and recurrent or metastatic squamous cell carcinoma of the head and neck progressing after platinum-based therapy.

Cetuximab is also FDA approved for the treatment of KRAS mutation-negative (wild-type) EGFR-expressing, metastatic colorectal cancer as determined by FDA approved tests, in particular as a first-line treatment in combination with FOLFIRI, or in combination with irinotecan in patients who are refractory to irinotecan-based chemotherapy, and for the treatment of patients who have failed oxaliplatin- and irinotecan-based chemotherapy or who are intolerant to irinotecan as a single agent.

ABX-EGF (VECTIBIX/panitumumab) is a human IgG2 antibody which, like cetuximab, binds to the extracellular domain of EGFR and competes with EGF for binding to EGFR, thereby inhibiting downstream pathway signaling and blocking proliferation of tumor cells (Voigt et al., 2012). Panitumumab is approved by the FDA for the treatment of patients with wild-type KRAS (exon 2 in codons 12 or 13) metastatic colorectal cancer (mCRC) as determined by an FDA-approved test, either as a first-line therapy in combination with FOLFOX or as a monotherapy following disease progression after prior treatment with fluoropyrimidine-, oxaliplatin-, and irinotecan-containing chemotherapy.

Necitumumab (Portrazza) is another antibody that binds EGFR and was approved by the FDA in 2015 for use in combination with gemcitabine and cisplatin for first-line treatment of patients with metastatic squamous non-small cell lung cancer.

Nimotuzumab (previously known as h-R3) is a humanized IgG1 antibody that binds to the extracellular region of EGFR which is enrolled in clinical trials in several countries. Nimotuzumab has been approved for treatment of squamous cell carcinoma in head and neck in India, Cuba, Argentina, Colombia, Ivory Coast, Gabon, Ukraine, Peru and Sri Lanka; as well as for the treatment of glioma (pediatric and adult) in Cuba, Argentina, Philippines and Ukraine; and for the treatment of nasopharyngeal cancer in China (Ramakrishnan et al., 2009).

Clinical testing of other antibodies targeting EGFR, including zalutuzumab (HuMax-EGFr) and matuzumab (formerly EMD 72000), has been initiated but these antibodies have not been granted regulatory approval and development has since stopped.

Antibody-drug conjugates (ADCs) advanced rapidly over the last years and were established as a permanent player in the field of oncology providing therapeutic benefit to patients suffering from various cancers. Consequently, five new ADCs were approved by the FDA between 2019 and August 2020 demonstrating the clinical success of this therapeutic class.¹⁻³ ADCs link the great selectivity of monoclonal antibodies with cell killing abilities of highly cytotoxic drugs and expand the therapeutic window by guiding these toxins to tumor cells. To date, approved ADCs and the vast majority of clinical and pre-clinical stage ADCs possess a monoclonal IgG scaffold.⁴ As a result of great success of conventional full-sized ADCs, alternative smaller antibody fragment-based drug conjugates are evolving.⁵ Such conjugates consist of Fab-fragments^{6,7}, single chain variable fragments (scFv)^{8,9}, diabodies¹⁰ or single-domain antibody-based structures like abdurins, nano-¹⁰ or humabodies¹³. Their small size allows better solid tumor penetration, due to elevated extravasation from blood vessels into the interstitial tissue space and interstitial diffusion through tissues.^{16,19} However, antibody fragments often do not show better efficacy^{7,13} which may relate to the absence of the Fc domain and its half-life extending function. The interaction of the Fc domain with its natural ligand, the neonatal Fc receptor (FcRn), mediates prolonged circulation of full-length IgG antibodies in the blood stream (e.g. mouse terminal t_{1/2} Trastuzumab vs. FcRn-nonbinding Trastuzumab 212 h vs. 6.9 h¹⁹). Therefore, fragments lacking the Fc portion are often hampered by fast systemic clearance rates and limited exposure (e.g. Trastuzumab Fab, mouse terminal t_{1/2} 4.4 h¹⁹). These findings led to a variety of novel conjugate formats in which small binder fragments were PASylated, fused to PEG¹⁰, albumin binding domains^{10,12,13} or Fc portions to improve their in vivo half-life, however, at the cost of increasing the hydrodynamic radius which limits the tumor penetration.

Therefore, ADCs show reduced solid tumor penetration due to their elevated size (150 kDa). This results in inhomogeneous exposition of cancer cells to cytotoxic doses of payload and a lower therapeutic efficacy of ADCs.

In contrast, the known smaller antibody fragment-based drug conjugates (≤50 kDa) show increased solid tumor penetration theoretically resulting in a more homogeneous exposition of cancer cells to the therapeutic. However, their smaller size and the lack of an FcRn binding site causes a shorter half-life of these fragment drug conjugates that counteracts a durable tumor penetration.

Thus, there remains the need to develop novel therapeutic options for the treatment of cancers by ADCs or antibody-fragment based conjugates which show an increased tumor penetration but at the same time a long half-life both mediating an increased therapeutic efficacy.

### SUMMARY OF THE INVENTION

Surprisingly, it has been found, that drug conjugates of another antibody-fragment based format, the Fc antigen binding fragment (Fcab), due to a smaller size and a Fc-mediated half-life extension, in contrast to the known ADCs and the known smaller antibody fragment-based drug conjugates, show at the same time both, an increased tumor penetration and a long half-life both mediating an increased therapeutic efficacy of such Fcab-drug conjugates. Accordingly, an efficient lysosomal delivery was observed for the EGFR Fcab-drug conjugates of the present invention resulting in potent cytotoxic effects in tumor cells. Thus, the EGFR Fcab-drug conjugates of the present invention can be used for the treatment of hyperproliferative diseases and disorders such as cancer.

Fcabs were never described or explored as anti-cancer drug conjugates. Fcabs were derived from the Fc fragment of human IgG1 antibodies by engineering the C-terminal structural loops of the C_{H}3 domain to form an antigen binding site (FIG. 3A, B). Hence, Fcabs combine Fc-mediated effector functions and neonatal Fc receptor (FcRn) binding with an antigen binding functionality but comprise only one third of the size of conventional IgGs.^{[15]} The smaller size of the Fcab format promises to improve solid tumor penetration by enhancing the extravasation from the circulation into the interstitial space and increasing diffusion rates through the interstitium and tumor tissue.^{[16]} Moreover, the half-life extending FcRn binding site delays systemic clearance of the Fcab (mouse terminal t_{1/2} Fcab 60-85 h^{[17,18]}) and thereby maintains a high plasma concentration that further drives penetration into tissues.^{[16]} FcRn binding provides Fcabs with a significant advantage over other reported ≤50 kDa Fab-^{[6,7]}, scFv-^{[8,9]}, diabody-^{[10]} or single domain antibody-based drug conjugates^{[11-13]} that lack an FcRn binding site and consequently suffer from a short half-life in vivo (mouse terminal t_{1/2} Trastuzumab-derived Fab 4.4 h^{[19]}). In similar experiments with HER Fcab-drug conjugates, we were able to show that the Fcab format is suitable to generate HER2 binding drug conjugates.^{[14]} Deploying an engineered microbial transglutaminase, we were able to site-specifically conjugate linker-payloads to the conserved Fc position Q295 resulting in stable and functional Fcab-drug conjugates with a drug-to-antibody ratio (DAR) of 2.0. Furthermore, we demonstrated in an in vitro spheroid experiment, that spheroid accumulation was higher for Fcabs compared to full-length antibody controls confirming the favorable penetration capability of Fcabs.^{[14]}

In the present experiments, we expanded the concept of Fcab-based ADCs from HER2 to EGFR binding Fcab-ADCs and demonstrated the versatility of this antibody format for the generation of site-specific, stable and highly potent drug conjugates. We first demonstrated that the selected EGFR binding Fcabs are suitable for an ADC approach based on selective cellular uptake data using heterogenous conjugates carrying a pH dependent dye. We then employed site-specific enzymatic conjugation to attach the microtubule inhibitor monomethyl auristatin E (MMAE) to position Q295 but also to the novel position Q311 and Q438 allowing to reach higher DARs. The drug conjugates showed retained EGFR and FcRn binding properties and possessed excellent stability in mouse and human serum. Finally, we showed EGFR-mediated sub-nanomolar cytotoxicity of our Fcab-drug conjugates on different cancer cell lines.

As shown herein, the favorable pharmacokinetic profile of Fcabs in combination with their small size surprisingly lead to a better and durable penetration of solid tumors by Fcab-based drug conjugates. This results in an elevated overall tumor exposure and better efficacy of the conjugates of the present invention in comparison to other fragment-based drug conjugates of similar size or conventional IgG-based ADCs (concept shown in FIG. 1).

Herein, we present for the first time the generation and functionality of EGFR targeting Fcab-drug conjugates. For proof of concept, we selected a diverse set of Fcabs that target the solid tumor antigen EGFR. As the intracellular release of the warhead is a prerequisite for an ADC, EGFR-dependent uptake rates for selected Fcab molecules were determined on cancer cells. Subsequently, various site-specific conjugation techniques were employed to couple Fcabs with the well-established tubulin inhibitor monomethyl auristatin E (MMAE). Moreover, target-dependent cytotoxicity and stability in serum were evaluated for all Fcab-drug conjugates as well as FcRn and target binding properties compared to parental Fcab molecules. Overall, the disclosed experiments and results emphasize the application of Fcabs for the generation of Fcab-drug-conjugates.

Fcabs produced in the scope of the present experiments bound EGFR with nanomolar affinity and accumulated target-dependently in EGFR expressing cells. Site-specific conjugation to Q295 and the novel positions Q311 and Q438 via mTG yielded DAR 2.7-2.9 Val-Cit-PAB-MMAE conjugates without altered EGFR or FcRn binding affinities. Generated Fcab-drug conjugates exhibited high stability in human and mouse serum and showed EGFR-mediated cytotoxicity at sub-nanomolar concentrations similar to Cetuximab-based reference conjugates.

Based on an extensive in vitro characterization, our experiments and results provide the proof-of-concept that the Fcab format is suitable for the generation of stable and cytotoxic drug conjugates. Moreover, in previous experiments with HER2 Fcab-drug conjugates we could demonstrate that the 50 kDa Fcab format shows superior penetration compared to a 150 kDa reference construct^{[14]}. The beneficial penetration of Fcab-drug conjugates demonstrates a better tumor penetration and an increase in overall tumor exposure and ultimately improved efficacy compared to ADCs.

Thus, the present invention relates to an EGFR Fcab-drug conjugate or a pharmaceutically acceptable salt thereof, comprising the formula Fcab-(L)ₘ-(D)ₙ wherein:
a) Fcab comprises an EGFR Fcab,
b) L comprises a linker,
c) D comprises a drug,
d) m is an integer from 1-5 and n is an integer from 1-10.

In a preferred embodiment of the present invention m is 1 to 3 and n is 1 to 5.

The present invention relates to an EGFR Fcab-drug conjugate according to the present invention wherein the EGFR Fcab is selected from the group consisting of: Fcab-1, Fcab-2, Fcab-3, Fcab-4, Fcab-5 and Fcab-6, having the amino acid sequences as set forth in SEQ ID Nos. 1-6.

A preferred embodiment of the present invention is an EGFR Fcab-drug conjugate according to the present invention wherein the EGFR Fcab is selected from the group consisting of: Fcab-1, Fcab-2 and Fcab-3, having the amino acid sequences as set forth in SEQ ID Nos. 1-3.

Also encompassed by the present invention are EGFR Fcab-drug conjugates according to the present invention wherein the amino acid sequence of the Fcabs is amended or modified by conservative amino acid substitutions. As used herein, the term “conservative substitution” refers to substitutions of amino acids which are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson, et al., MOLECULAR BIOLOGY OF THE GENE, The Benjamin/Cummings Pub. Co., p. 224 (4th Edition 1987)).

In general, any drug can be conjugated to the EGFR Fcab-drug conjugate obtained according to the inventive method, as long as it is preferably sufficiently stable to prevent its premature release before reaching the desired target cell, thereby preventing damage to non-target cells and increasing availability at the target site. As the drug is most commonly released in the lysosome following cleavage of the linker molecule, it is important to ensure that the drug remains stable in low pH environments and has the capacity to move into the cytosolic or nuclear compartments of the cell where it takes effect. Similarly, it is desirable that the molecular structure of the drug allows for its conjugation to the linker while avoiding immunogenicity, maintaining the internalization rate of the EGFR Fcab-drug conjugate and promoting or at least not compromising its biological effects, if any (i.e., ADCC, CDCC and CDC). Regardless of the stability of the drug, only a small portion of the administered EGFR Fcab-drug conjugate will typically reach the target cells. Thus, the conjugated drug is preferably potent at low concentrations.

Thus, one embodiment of the present invention is an EGFR Fcab-drug conjugate, wherein the EGFR Fcab is conjugated to a drug selected from a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate). The use of antibody-drug conjugates as ADCs and the EGFR Fcab-drug conjugates of the present invention for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg. Del. Rev. 26:151-172; U.S. Pat. No. 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al. (2000) Jour. of the Nat. Cancer Inst. 92(19):1573-1581; Mandler et al. (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al. (1998) Cancer Res. 58:2928; Hinman et al. (1993) Cancer Res. 53:3336-3342). The toxins may assert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Suitable drugs envisaged for preparing the EGFR Fcab-drug conjugates of the invention include all cytotoxins commonly utilized in ADCs to date. Most classes of cytotoxins act to inhibit cell division and are classified based on their mechanism of action. Exemplary cytotoxins that are conceivable as part of the inventive EGFR Fcab-drug conjugates include, without limitation, anthracycline, doxorubicin, methotrexate, auristatins including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers.

In one embodiment, the drug moiety is selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor. In some embodiments, the cytotoxic agent is a maytansinoid, wherein the maytansinoid is N(2′)-deacetyl-N(2′)-(3-mercapto-I-oxopropyl)-maytansine (DM1), N(2′)-deacetyl-N(2′)-(4-mercapto-I-oxopentyl)-maytansine (DM3) or N(2′)-deacetyl-N2-(4-mercapto-4-methyl-1-oxopentyl)-maytansine (DM4).

Thus, a preferred embodiment of the present invention is the EGFR Fcab-drug conjugate of the present invention wherein the drug is selected from the group consisting of: anthracycline, doxorubicin, methotrexate, an auristatin including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers, a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In a particular preferred embodiment, the drug is the tubulin inhibitor monomethyl auristatin E (MMAE).

Linkers are preferably designed to be stable in the blood stream (to conform to the increased circulation time of antibodies) and labile at the target site to allow rapid release of the drug. Parameters taken into consideration when designing a suitable linker typically include cleavability of the linker and the position and mechanism of linkage (i.e. conjugation chemistry). Existing linkers are traditionally classified as cleavable or non-cleavable linkers.

Cleavable linkers exploit the change in environment upon internalization of the EGFR Fcab-antigen complex into target cells, resulting in cleavage of the linker and release of the drug into the target cell. Exemplary cleavable linkers that are contemplated for use with the EGFR Fcab drug conjugates provided herein include hydrazone, disulfide and peptide linkers. In contrast to cleavable linkers that rely on distinctive intracellular conditions to release the drug, non-cleavable linkers such as thioether linkers depend solely on the process of proteolytic degradation following EGFR Fcab-antigen internalization and processing in the lysosomal pathway. Linkers for antibody-drug design are well-known in the art and have been reviewed, i.e., by Peters and Brown, Biosci. Rep. 2015 August; 35(4): e00225. One or several drugs can be linked to each EGFR Fcab in order to achieve adequate therapeutic efficacy.

Means and methods for preparing ADCs are described in the art and have been reviewed, i.e., by Peters and Brown (supra). Traditionally, drugs are chemically conjugated to antibodies using conventional techniques, whereby reactive portions of native amino acids are made to interact and bind a specific part of the linker molecule. Examples of reactive groups include the epsilon-amino end of lysine residues and the thiol side chains present in the partially reduced form of cysteine residues. Alternatives to conventional conjugation techniques include conjugation via (i) novel unpaired cysteine residues introduced at specific, controlled sites along the antibody using site-directed mutagenesis, (ii) microbial transglutaminases that recognize glutamine ‘tag’ sequences that can be incorporated into the antibody via plasmids, adding amine-containing drugs to the glutamine side chains, or (iii) non-natural amino acids, such as selenocysteine or acetylphenylalanine introduced into the antibody during transcription, that are available for conjugation with a suitable cytotoxin, for instance in the case of nucleophilic selenocysteine, a positively charged drug molecule.

The drug moiety D can be linked to the EGFR Fcab through linker L. L is any chemical moiety capable of linking the drug moiety to the antibody through covalent bonds. A cross-linking reagent is a bifunctional or multifunctional reagent that can be used to link a drug moiety and an Fcab to form an EGFR Fcab-drug conjugate. EGFR Fcab drug conjugates can be prepared using a cross-linking reagent having a reactive functionality capable of binding to both the drug moiety and the EGFR Fcab. For example, a cysteine, thiol or an amine, e.g. N-terminus or an amino acid side chain, such as lysine of the EGFR Fcab, can form a bond with a functional group of a cross-linking reagent.

In one embodiment, L is a cleavable linker. In another embodiment, L is a non-cleavable linker. In some embodiments, L is an acid-labile linker, photo-labile linker, peptidase cleavable linker, esterase cleavable linker, a disulfide bond cleavable linker, a hydrophilic linker, a procharged linker, or a dicarboxylic acid-based linker.

Suitable cross-linking reagents that form a non-cleavable linker between the drug moiety, for example may tansinoid, and the antibody are well known in the art, and can form non-cleavable linkers that comprise a sulfur atom (such as SMCC) or those that are without a sulfur atom. Preferred cross-linking reagents that form non-cleavable linkers between the drug moiety, for example maytansinoid, and the EGFR Fcab comprises a maleimido- or haloacetyl-based moiety. According to the present invention, such non-cleavable linkers are said to be derived from maleimido- or haloacetyl based moieties.

Cross-linking reagents comprising a maleimido based moiety include but not limited to, N-succinimidyl-4-(maleimidomethyl)cyclohexanecarboxylate (SMCC), sulfo-succinimidyl 4-(N-maleimidomethyl)cyclohexane-1-carboxylate (sulfo-SMCC), N-succinimidyl-4-(maleimidomethyl)cyclohexane-1-carboxy-(6-amidocaproate), which is a “long chain” analog of SMCC (LC-SMCC), K-maleimidoundeconoic acid N-succinimidylester (KMUA), Y-maleimidobutyric acid N-succinimidylester (GMBS), e-maleimidocaproic acid N-succinimidylester (EMCS), m-maleimidobenzoyl-N-hydroxysuccinimideester (MBS), N—O-maleimidoacetoxy)-succinimide ester (AMSA), succinimidyl-6-(B-maleimidopropionamido) hexanoate (SMPH), N-succinimidyl-4-(p-maleimidophenyl)-butyrate (SMPB), N-(-p-maleomidophenyl)-isocyanate (PMIP) and maleimido-based cross-linking reagents containing a polyethylhene glycol spacer, such as MAL-PEG-NHS. These cross-linking reagents form non-cleavable linkers derived from maleimido-based moieties.

Thus, a preferred embodiment of the present invention is an EGFR Fcab-drug conjugate of the present invention wherein the linker is selected from the linkers described herein.

Another preferred embodiment of the present invention is an EGFR Fcab-drug conjugate of the present invention wherein the linker is selected from the group consisting of an acid-labile linker, a photo-labile linker, a peptidase cleavable linker, an esterase cleavable linker, a disulfide bond cleavable linker, a hydrophilic linker, a procharged linker and a dicarboxylic acid-based linker.

A further preferred embodiment of the present invention is an EGFR Fcab-drug conjugate of the present invention wherein the linker is a disulfide bond cleavable linker.

Each of the embodiments described herein can be combined with any other embodiment described herein not inconsistent with the embodiment with which it is combined. Furthermore, unless incompatible in a given context, wherever a compound is stipulated which is capable of ionization (e.g. protonation or deprotonation), the definition of said compound includes any pharmaceutically acceptable salts thereof. Accordingly, the phrase “or a pharmaceutically acceptable salt thereof” is implicit in the description of all compounds described herein. Embodiments within an aspect as described below can be combined with any other embodiments not inconsistent within the same aspect or a different aspect. For instance, embodiments of any of the treatment methods of the present invention can be combined with any embodiments of the combination products of the present invention or pharmaceutical composition of the present invention, and vice versa. Likewise, any detail or feature given for the treatment methods of the present invention apply—if not inconsistent—to those of the combination products of the present invention and pharmaceutical compositions of the present invention, and vice versa.

The present invention may be understood more readily by reference to the detailed description above and below of the particular and preferred embodiments of the invention and the examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

The term “about” when used to modify a numerically defined parameter refers to any minimal alteration in such parameter that does not change the overall effect, e.g., the efficacy of the agent in treatment of a disease or disorder. In some embodiments, the term “about” means that the parameter may vary by as much as 10% below or above the stated numerical value for that parameter.

“Administering” or “administration of” a drug to a patient (and grammatical equivalents of this phrase) refers to direct administration, which may be administration to a patient by a medical professional or may be self-administration, and/or indirect administration, which may be the act of prescribing a drug, e.g., a physician who instructs a patient to self-administer a drug or provides a patient with a prescription for a drug is administering the drug to the patient.

An “amino acid difference” refers to a substitution, a deletion or an insertion of an amino acid.

“Antibody” is an immunoglobulin (Ig) molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, as well as any protein comprising such antigen-binding fragment or antibody fragment thereof, including fusion proteins (e.g., antibody-drug conjugates, an antibody fused to a cytokine or an antibody fused to a cytokine receptor), antibody compositions with poly-epitopic specificity, and multi-specific antibodies (e.g., bispecific antibodies). The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, C T, 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ε, γ and μ, respectively. The γ and α classes are further divided into subclasses based on relatively minor differences in the C_{H} sequence and function, e.g., humans express the following subclasses: IgG1, IgG2A, IgG2B, IgG3, IgG4, IgA1, and IgK1.

“Antigen-binding fragment” of an antibody or “antibody fragment” comprises a portion of an intact antibody, which is still capable of antigen binding. Antigen-binding fragments include, for example, Fab, Fab′, F(ab′)2, Fd, Fcab and Fv fragments, domain antibodies (dAbs, e.g., shark and camelid antibodies), fragments including CDRs, single chain variable fragment antibodies (scFv), single-chain antibody molecules, multi-specific antibodies formed from antibody fragments, maxibodies, nanobodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8HO: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy, e.g., EGFR profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

By “cancer” is meant a collection of cells multiplying in an abnormal manner. As used herein, the term “cancer” refers to all types of cancer, neoplasm, malignant or benign tumors found in mammals, including leukemia, carcinomas, and sarcomas. Exemplary cancers include acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

“CDRs” are the complementarity determining region amino acid sequences of an antibody, antibody fragment or antigen-binding fragment. These are the hypervariable regions of immunoglobulin heavy and light chains. There are three heavy chain and three light chain CDRs (or CDR regions) in the variable portion of an immunoglobulin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Combination” as used herein refers to the provision of a first active modality in addition to one or more further active modalities (wherein one or more active modalities may be fused). Contemplated within the scope of the combinations described herein, are any regimen of combination modalities or partners (i.e., active compounds, components or agents), encompassed in single or multiple compounds and compositions. It is understood that any modalities within a single composition, formulation or unit dosage form (i.e., a fixed-dose combination) must have the identical dose regimen and route of delivery. It is not intended to imply that the modalities must be formulated for delivery together (e.g., in the same composition, formulation or unit dosage form). The combined modalities can be manufactured and/or formulated by the same or different manufacturers. The combination partners may thus be, e.g., entirely separate pharmaceutical dosage forms or pharmaceutical compositions that are also sold independently of each other.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method.

“Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Drug conjugate” or “drug” according to the present invention is a conjugate of an EGFR Fcab according to the present invention and a drug selected from the group including but not limited to anthracycline, doxorubicin, methotrexate, an auristatin including monomethyl auristatin E (MMAE) and monomethyl auristatin F (MMAF), maytansines and their maytansinoids derivatives (DMs), calicheamicins, duocarymycins and pyrrolobenzodiazepine (PBD) dimers, a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an amanitin, a pyrrolobenzodiazepine, an RNA polymerase inhibitor, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder or a DHFR inhibitor.

“Fcab” according to the present invention is an IgG1-based homodimeric Fc region that combine Fc effector functions with an engineered antigen binding site located at the C-terminal structural loops in the C_{H}3 domain.²¹⁻²³. Antigen-binding Fc fragments (also referred to as Fcab™ [Fc fragment with antigen binding]) comprising e.g., a modified IgG1 Fc domain which binds to EGFR with high affinity, are described in WO 2009/132876 A 1 and WO 2009/000006 A 1 which are hereby incorporated by reference in their entirety. Specific binding members described herein include antigen binding Fc fragments described herein which each has one or more amino acid modifications in at least one structural loop region, wherein the modified structural loop region specifically binds to an epitope of an antigen, e.g. EGFR, to which an unmodified Fc fragment does not significantly bind.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells. Antigen-binding Fc fragments may comprise an antigen-binding site engineered into one or more structural loop regions of a constant domain of the Fc fragment, e.g. the C_{H}2 or C_{H}3 domain. The preparation of antigen-binding Fc fragments is described in WO 2006/072620 and WO2009/132876. A specific binding member for use in the present invention preferably is, or comprises, an antigen binding Fc fragment, also referred to as Fcab™. More preferably, a specific binding member for use in the present invention is an antigen-binding Fc fragment. The specific binding member may be an IgA1, IgA2, IgD, IgE, IgG, IgG2, IgG3, IgG4 or IgM antigen-binding Fc fragment. Most preferably, a specific binding member as referred to herein is an IgG1 (e.g., human IgG1) antigen-binding Fc fragment. In certain embodiments, a specific binding member is an IgG1 antigen-binding Fc fragment comprising a hinge or portion thereof, a C_{H}2 domain and a C_{H}3 domain.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol. 147(I): 86; van Dijk and van de Winkel (2001) Curr. Opin. Pharmacol. 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6, 150,584 regarding XENOMOUSE technology). See also, for example, Li et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“Infusion” or “infusing” refers to the introduction of a drug-containing solution into the body through a vein for therapeutic purposes. Generally, this is achieved via an intravenous (IV) bag.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and U.S. Pat. No. 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93).

The monoclonal antibodies herein specifically include chimeric antibodies (immunoglobulins) in which a portion of the heavy and/or light chain is identical to or homologous to corresponding sequences in antibodies derived from a particular species or belonging to a particular antibody class or subclass, while the remainder of the chain(s) is (are) identical to or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used, e.g., in screening, characterizing, and evaluating drugs and therapies.

“Percent (%) sequence identity” with respect to a peptide or polypeptide sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2 or ALIGN software. Those skilled in the art can determine appropriate parameters for measuring alignment, including any algorithms needed to achieve maximal alignment over the full length of the sequences being compared.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Pharmaceutically acceptable salt” forms of EGFR Fcab-drug conjugate are for the most part prepared by conventional methods. If the EGFR Fcab-drug conjugate of the present invention contains a carboxyl group, one of its suitable salts can be formed by reacting the compound of the present invention with a suitable base to give the corresponding base-addition salt. Such bases are, for example, alkali metal hydroxides, including potassium hydroxide, sodium hydroxide and lithium hydroxide; alkaline-earth metal hydroxides, such as barium hydroxide and calcium hydroxide; alkali metal alkoxides, for example potassium ethoxide and sodium propoxide; and various organic bases, such as piperidine, diethanolamine and N-methylglutamine.

Furthermore, the base salts of the EGFR Fcab-drug conjugate of the present invention include aluminium, ammonium, calcium, copper, iron(III), iron(II), lithium, magnesium, manganese(III), manganese(II), potassium, sodium and zinc salts, but this is not intended to represent a restriction.

Of the above-mentioned salts, preference is given to ammonium; the alkali metal salts sodium and potassium, and the alkaline-earth metal salts calcium and magnesium. Salts of the EGFR Fcab-drug conjugate of the present invention which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)methylamine (tromethamine), but this is not intended to represent a restriction.

As mentioned, the pharmaceutically acceptable base-addition salts of EGFR Fcab-drug conjugate are formed with metals or amines, such as alkali metals and alkaline-earth metals or organic amines. Preferred metals are sodium, potassium, magnesium and calcium. Preferred organic amines are N, N′-dibenzylethylene-diamine, chloroprocaine, choline, diethanolamine, ethylenediamine, N-methyl-D-glucamine and procaine.

The base-addition salts of the EGFR Fcab-drug conjugate of the present invention are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

“Prodrug” refers to derivatives of the EGFR Fcab-drug conjugates of the present invention which have been modified by means of, for example, alkyl or acyl groups (see also amino- and hydroxyl-protecting groups below), sugars or oligopeptides and which are rapidly cleaved or liberated in the organism to form the effective molecules. These also include biodegradable polymer derivatives of the EGFR Fcab-drug conjugate of the present invention, as described, for example, in Int. J. Pharm. 115 (1995), 61-67.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Single-chain Fv”, also abbreviated as “sFv” or “scFv”, are antibody fragments that comprise the V_{H} and V_{L} antibody domains connected into a single polypeptide chain. In some embodiments, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

“Solvates” refer to adductions of inert solvent molecules onto the EGFR Fcab-drug conjugates of the invention which form owing to their mutual attractive force. Solvates are, for example, hydrates, such as monohydrates or dihydrates, or alcoholates, i.e. addition compounds with alcohols, such as, for example, with methanol or ethanol.

By “substantially identical” is meant (1) a query amino acid sequence exhibiting at least 75%, 85%, 90%, 95%, 99% or 100% amino acid sequence identity to a subject amino acid sequence or (2) a query amino acid sequence that differs in not more than 20%, 30%, 20%, 10%, 5%, 1% or 0% of its amino acid positions from the amino acid sequence of a subject amino acid sequence and wherein a difference in an amino acid position is any of a substitution, deletion or insertion of an amino acid.

“Systemic” treatment is a treatment, in which the drug substance travels through the bloodstream, reaching and affecting cells all over the body.

“Therapeutically effective amount” of EGFR Fcab-drug conjugate, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose and may occur only after administration of a series of doses. Thus, a therapeutically effective amount may be administered in one or more administrations. Such therapeutically effective amount may vary according to factors such as the disease state, age, sex, and weight of the individual, and the ability of an EGFR Fcab-drug conjugate to elicit a desired response in the individual. A therapeutically effective amount is also one in which any toxic or detrimental effects of an EGFR Fcab-drug conjugate are outweighed by the therapeutically beneficial effects. The term “effective amount” denotes the amount of a medicament or of a pharmaceutical active compound which causes in a tissue, system, animal or human a biological or medical response which is sought or desired, for example, by a researcher or physician.

In addition, the term “therapeutically effective amount” denotes an amount which, compared with a corresponding subject who has not received this amount, has the following consequence: improved treatment, healing, prevention or elimination of a disease, syndrome, disease state, complaint, disorder or prevention of side effects or also a reduction in the progress of a disease, complaint or disorder. The term “therapeutically effective amount” also encompasses the amounts which are effective for increasing normal physiological function.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

When discovering and developing therapeutic agents, the person skilled in the art attempts to optimise pharmacokinetic parameters while retaining desirable in-vitro properties. It is reasonable to assume that many compounds with poor pharmacokinetic profiles are susceptible to oxidative metabolism. In-vitro liver microsomal assays currently available provide valuable information on the course of oxidative metabolism of this type, which in turn permits the rational design of deuterated compounds of the present invention with improved stability through resistance to such oxidative metabolism. Significant improvements in the pharmacokinetic profiles of the EGFR Fcab-drug conjugates of the present invention are thereby obtained and can be expressed quantitatively in terms of increases in the in-vivo half-life (T/2), concentration at maximum therapeutic effect (Cₘₐₓ), area under the dose response curve (AUC), and F; and in terms of reduced clearance, dose and costs of materials.

The invention also relates, in particular, to a medicament comprising at least one EGFR Fcab-drug conjugate according to the invention for use in the treatment and/or prophylaxis of physiological and/or pathophysiological states.

Physiological and/or pathophysiological states are taken to mean physiological and/or pathophysiological states which are medically relevant, such as, for example, diseases or illnesses and medical disorders, complaints, symptoms or complications and the like, in particular diseases.

A preferred embodiment of the present invention is a medicament comprising at least one EGFR Fcab-drug conjugate according to the present invention for use in the treatment and/or prophylaxis of physiological and/or pathophysiological states, selected from the group consisting of hyperproliferative diseases and disorders.

A yet more preferred embodiment of the present invention is a medicament according to the present invention for use in the treatment and/or prophylaxis of physiological and/or pathophysiological states, selected from the group consisting of hyperproliferative diseases and disorders, wherein the hyperproliferative disease or disorder is cancer.

Another preferred embodiment of the present invention is a medicament according to the present invention for use in the treatment of cancer, wherein the cancer is selected from the group consisting of acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

Preference is given, in particular, to physiological and/or pathophysiological states which are connected to EGFR. Thus, the present invention relates to a medicament according to the present invention for use in the treatment of EGFR-positive cancers.

A cancer as referred to herein may be a gastric cancer, breast cancer, colorectal cancer, ovarian cancer, pancreatic cancer, lung cancer (for example, non-small cell lung cancer), stomach cancer, or endometrial cancer. All of these cancers have been shown to overexpress EGFR. Preferably, the cancer is gastric cancer, breast cancer, or colorectal cancer. More preferably, the cancer is gastric cancer or breast cancer. In one preferred embodiment, the cancer is gastric cancer. Gastric cancer, as referred to herein, includes esophageal cancer. In another preferred embodiment, the cancer is breast cancer. The EGFR gene copy number of the cancer is as set out above. Such a cancer may be referred to as EGFR-positive (EGFR+) or as overexpressing EGFR. Thus, a cancer, as referred to herein, may be EGFR-positive. In addition, or alternatively, a cancer as referred to herein may overexpress EGFR. Whether a cancer is EGFR-positive or overexpresses EGFR may, for example, be determined initially using immunohistochemistry (IHC), optionally followed by methods such as qPCR as outlined above.

A further preferred embodiment is a medicament according to the present invention for use in the treatment solid cancers including breast cancer, gastric cancer, stomach cancer, colorectal cancer, ovarian cancer, pancreatic cancer, endometrial cancer or non-small cell lung cancer.

In an preferred embodiment the cancer is selected from the group consisting of lung cancer, for example non-small cell lung cancer [NSCLC]), glioblastoma multiforme, skin cancer, for example cutaneous squamous cell carcinoma, head and neck cancer such as head and neck squamous-cell carcinoma [HNSCC]), breast cancer, stomach cancer (gastric cancer), colorectal cancer (CRC), ovarian cancer, pancreatic cancer and endometrial cancer.

It is intended that the medicaments disclosed above include a corresponding use of the EGFR Fcab-drug conjugate according to the invention for the preparation of a medicament for the treatment and/or prophylaxis of the above physiological and/or pathophysiological states.

It is additionally intended that the medicaments disclosed above include a corresponding method for the treatment and/or prophylaxis of the above physiological and/or pathophysiological states in which at least one EGFR Fcab-drug conjugate according to the invention is administered to a patient in need of such a treatment.

Accordingly, also an embodiment of the present invention is the use of an EGFR Fcab-drug conjugate according to the present invention for the treatment of cancer.

Accordingly, also an embodiment of the present invention is the use of an EGFR Fcab-drug conjugate for the manufacture of a medicament for the treatment of cancer.

Accordingly, also an embodiment of the present invention is a method for treating cancer in a subject wherein the method comprises administering the EGFR Fcab-drug conjugate or the pharmaceutical preparation according to the present invention to the subject.

Accordingly, also an embodiment of the present invention is the use of a method for the treatment of cancer comprising administering the EGFR Fcab-drug conjugate or the pharmaceutical preparation according to the present invention to a subject in need thereof.

In one embodiment, the EGFR Fcab-drug conjugate of the invention is used in the treatment of a human subject. The main expected benefit in the treatment with the therapeutic combination of the EGFR Fcab and the drug is a gain in risk/benefit ratio for these human patients. The administration of the EGFR Fcab-drug conjugates of the invention may be advantageous over the individual therapeutic agents in that the combinations of the EGFR Fcab and the drug may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, and/or vi) an increase in the bioavailability of one or both of the therapeutic agents.

In certain embodiments, the invention provides for the treatment of diseases, disorders, and conditions characterized by excessive or abnormal cell proliferation. Such diseases include a proliferative or hyperproliferative disease. Examples of proliferative and hyperproliferative diseases include cancer and myeloproliferative disorders.

In another embodiment, the cancer is selected from carcinoma, lymphoma, leukemia, blastoma, and sarcoma. More particular examples of such cancers include squamous cell carcinoma, myeloma, small-cell lung cancer, non-small cell lung cancer, glioma, Hodgkin's lymphoma, non-Hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma, cervical cancer, brain cancer, stomach cancer, bladder cancer, hepatoma, breast cancer, colon carcinoma, biliary tract cancer, and head and neck cancer. The disease or medical disorder in question may be selected from any of those disclosed in WO2015118175, WO2018029367, WO2018208720, PCT/US18/12604, PCT/US19/47734, PCT/US19/40129, PCT/US19/36725, PCT/US19/732271, PCT/US19/38600, PCT/EP2019/061558.

In one embodiment, the cancer is selected from: appendiceal cancer, bladder cancer, breast cancer, cervical cancer, colorectal cancer, endometrial cancer, esophageal cancer (in particular esophageal squamous cell carcinoma), fallopian tube cancer, gastric cancer, glioma (such as diffuse intrinsic pontine glioma), head and neck cancer (in particular head and neck squamous cell carcinoma and oropharyngeal cancer), leukemia (in particular acute lymphoblastic leukemia, acute myeloid leukemia) lung cancer (in particular non-small cell lung cancer), lymphoma (in particular Hodgkin's lymphoma, non-Hodgkin's lymphoma), melanoma, mesothelioma (in particular malignant pleural mesothelioma), Merkel cell carcinoma, neuroblastoma, oral cancer, osteosarcoma, ovarian cancer, prostate cancer, renal cancer, salivary gland tumor, sarcoma (in particular Ewing's sarcoma or rhabdomyosarcoma) squamous cell carcinoma, soft tissue sarcoma, thymoma, thyroid cancer, urothelial cancer, uterine cancer, vaginal cancer, vulvar cancer or Wilms tumor. In a further embodiment, the cancer is selected from: appendiceal cancer, bladder cancer, cervical cancer, colorectal cancer, esophageal cancer, head and neck cancer, melanoma, mesothelioma, non-small-cell lung cancer, prostate cancer and urothelial cancer. In a further embodiment, the cancer is selected from cervical cancer, endometrial cancer, head and neck cancer (in particular head and neck squamous cell carcinoma and oropharyngeal cancer), lung cancer (in particular non-small cell lung cancer), lymphoma (in particular non-Hodgkin's lymphoma), melanoma, oral cancer, thyroid cancer, urothelial cancer or uterine cancer. In another embodiment, the cancer is selected from head and neck cancer (in particular head and neck squamous cell carcinoma and oropharyngeal cancer), lung cancer (in particular non-small cell lung cancer), urothelial cancer, melanoma or cervical cancer.

In one embodiment, the human has a solid tumor. In one embodiment, the solid tumor is advanced solid tumor. In one embodiment, the cancer is selected from head and neck cancer, squamous cell carcinoma of the head and neck (SCCHN or HNSCC), gastric cancer, melanoma, renal cell carcinoma (RCC), esophageal cancer, non-small cell lung carcinoma, prostate cancer, colorectal cancer, ovarian cancer and pancreatic cancer. In one embodiment, the cancer is selected from the group consisting of: colorectal cancer, cervical cancer, bladder cancer, urothelial cancer, head and neck cancer, melanoma, mesothelioma, non-small cell lung carcinoma, prostate cancer, esophageal cancer, and esophageal squamous cell carcinoma. In one aspect the human has one or more of the following: SCCHN, colorectal cancer, esophageal cancer, cervical cancer, bladder cancer, breast cancer, head and neck cancer, ovarian cancer, melanoma, renal cell carcinoma (RCC), esophageal squamous cell carcinoma, non-small cell lung carcinoma, mesothelioma (e.g. pleural malignant mesothelioma), and prostate cancer. In another aspect the human has a liquid tumor such as diffuse large B cell lymphoma (DLBCL), multiple myeloma, chronic lymphoblastic leukemia, follicular lymphoma, acute myeloid leukemia and chronic myelogenous leukemia.

In some embodiments, the cancer is an advanced cancer. In some embodiments, the cancer is a metastatic cancer. In some embodiments, the cancer is a recurrent cancer (e.g. a recurrent gynecological cancer such as recurrent epithelial ovarian cancer, recurrent fallopian tube cancer, recurrent primary peritoneal cancer, or recurrent endometrial cancer). In one embodiment, the cancer is recurrent or advanced.

In various embodiments, the method of the invention is employed as a first, second, third or later line of treatment. A line of treatment refers to a place in the order of treatment with different medications or other therapies received by a patient. First line therapy regimens are treatments given first, whereas second- or third-line therapy is given after the first line therapy or after the second line therapy, respectively. Therefore, first line therapy is the first treatment for a disease or condition. In patients with cancer, first line therapy, sometimes referred to as primary therapy or primary treatment, can be surgery, chemotherapy, radiation therapy, or a combination of these therapies. Typically, a patient is given a subsequent chemotherapy regimen (second or third line therapy), either because the patient did not show a positive clinical outcome or only showed a sub-clinical response to a first or second line therapy or showed a positive clinical response but later experienced a relapse, sometimes with disease now resistant to the earlier therapy that elicited the earlier positive response.

In some embodiments, the treatment of cancer is first line treatment of cancer. In one embodiment, the treatment of cancer is second line treatment of cancer. In some embodiments, the treatment is third line treatment of cancer. In some embodiments, the treatment is fourth line treatment of cancer. In some embodiments, the treatment is fifth line treatment of cancer. In some embodiments, prior treatment to said second line, third line, fourth line or fifth line treatment of cancer comprises one or more of radiotherapy, chemotherapy, surgery or radiochemotherapy.

In one embodiment, the prior treatment comprises treatment with diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; nivolumab; FOLFOX; capecitabine; FOLFIRI; bevacizumab; atezolizumab; selicrelumab; obinotuzumab or any combinations thereof. In one embodiment, prior treatment to said second line treatment, third line, fourth line or fifth line treatment of cancer comprises ipilimumab and nivolumab. In one embodiment, prior treatment to said second line treatment, third line, fourth line or fifth line treatment of cancer comprises FOLFOX, capecitabine, FOLFIRI/bevacizumab and atezolizumab/selicrelumab. In one embodiment, prior treatment to said second line treatment, third line, fourth line or fifth line treatment of cancer comprises carboplatin/Nab-paclitaxel. In one embodiment, prior treatment to said second line treatment, third line, fourth line or fifth line treatment of cancer comprises nivolumab and electrochemotherapy. In one embodiment, prior treatment to said second line treatment, third line, fourth line or fifth line treatment of cancer comprises radiotherapy, cisplatin and carboplatin/paclitaxel.

In one embodiment, the methods of the present invention further comprise administering at least one neo-plastic agent or cancer adjuvant to said human. The methods of the present invention may also be employed with other therapeutic methods of cancer treatment.

Typically, any anti-neoplastic agent or cancer adjuvant that has activity versus a tumor, such as a susceptible tumor being treated may be co-administered in the treatment of cancer in the present invention. Examples of such agents can be found in Cancer Principles and Practice of Oncology by V. T. Devita, T. S. Lawrence, and S. A. Rosenberg (editors), 10th edition (Dec. 5, 2014), Lippincott Williams & Wilkins Publishers.

In one embodiment, the human has previously been treated with one or more different cancer treatment modalities. In some embodiments, at least some of the patients in the cancer patient population have previously been treated with one or more therapies, such as surgery, radiotherapy, chemotherapy or immunotherapy. In some embodiments, at least some of the patients in the cancer patient population have previously been treated with chemotherapy (e.g. platinum-based chemotherapy). For example, a patient who has received two lines of cancer treatment can be identified as a 2 L cancer patient (e.g. a 2 L NSCLC patient). In some embodiments, a patient has received two lines or more lines of cancer treatment (e.g. a 2 L+ cancer patient such as a 2 L+ endometrial cancer patient). In some embodiments, a patient has not been previously treated with an antibody therapy, such as an anti-PD-1 therapy. In some embodiments, a patient previously received at least one line of cancer treatment (e.g. a patient previously received at least one line or at least two lines of cancer treatment). In some embodiments, a patient previously received at least one line of treatment for metastatic cancer (e.g. a patient previously received one or two lines of treatment for metastatic cancer).

The EGFR Fcab-drug conjugates according to the invention preferably exhibit an advantageous biological activity which can easily be demonstrated in enzyme assays and animal experiments, as described in the examples. In such enzyme-based assays, the EGFR Fcab-drug conjugates according to the invention preferably exhibit and cause an inhibiting effect, which is usually documented by IC₅₀ values in a suitable range, preferably in the micromolar range and more preferably in the nanomolar range.

The EGFR Fcab-drug conjugates of the present invention can be used for the preparation of pharmaceutical preparations, in particular by non-chemical methods. In this case, they are brought into a suitable dosage form together with at least one solid, liquid and/or semi-liquid excipient or adjuvant and optionally in combination with one or more further active compound(s).

Thus, the invention further relates to a pharmaceutical preparation comprising EGFR Fcab-drug conjugate according to the present invention.

In another embodiment of the present invention this pharmaceutical preparation comprises further excipients and/or adjuvants. Additionally, another embodiment according to the present invention is a pharmaceutical preparation which comprises at least one EGFR Fcab-drug conjugate according to the present invention and at least one further medicament active compound.

The invention further relates to a process for the preparation of a pharmaceutical preparation, characterised in that an EGFR Fcab-drug conjugate according to the present invention is brought into a suitable dosage form together with a solid, liquid or semi-liquid excipient or adjuvant.

The pharmaceutical preparations according to the invention can be used as medicaments in human or veterinary medicine and can be used in the therapeutic treatment of the human or animal body and in the combating of the above-mentioned diseases. The patient or host can belong to any mammal species, for example a primate species, particularly humans; rodents, including mice, rats and hamsters; rabbits; horses, cattle, dogs, cats, etc. Animal models are of interest for experimental investigations, where they provide a model for the treatment of a human disease. They can furthermore be used as diagnostic agents or as reagents.

Suitable carrier substances are organic or inorganic substances which are suitable for enteral (for example oral), parenteral or topical administration and do not react with the novel compounds, for example water, vegetable oils (such as sunflower oil or cod-liver oil), benzyl alcohols, polyethylene glycols, gelatine, carbohydrates, such as lactose or starch, magnesium stearate, talc, lanolin or Vaseline. Owing to his expert knowledge, the person skilled in the art is familiar with which adjuvants are suitable for the desired medicament formulation. Besides solvents, for example water, physiological saline solution or alcohols, such as, for example, ethanol, propanol or glycerol, sugar solutions, such as glucose or mannitol solutions, or a mixture of the said solvents, gel formers, tablet assistants and other active-ingredient carriers, it is also possible to use, for example, lubricants, stabilisers and/or wetting agents, emulsifiers, salts for influencing the osmotic pressure, antioxidants, dispersants, antifoams, buffer substances, flavours and/or aromas or flavour correctants, preservatives, solubilizers or dyes. If desired, preparations or medicaments according to the invention may comprise one or more further active compounds, for example one or more vitamins.

If desired, preparations or medicaments according to the invention may comprise one or more further active compounds and/or one or more action enhancers (adjuvants).

The terms “pharmaceutical formulation” and “pharmaceutical preparation” are used as synonyms for the purposes of the present invention.

As used here, “pharmaceutically tolerated” relates to medicaments, precipitation reagents, excipients, adjuvants, stabilisers, solvents and other agents which facilitate the administration of the pharmaceutical preparations obtained therefrom to a mammal without undesired physiological side effects, such as, for example, nausea, dizziness, digestion problems or the like.

In pharmaceutical preparations for parenteral administration, there is a requirement for isotonicity, euhydration and tolerability and safety of the formulation (low toxicity), of the adjuvants employed and of the primary packaging. Surprisingly, the EGFR Fcab-drug conjugates according to the present invention preferably have the advantage that direct use is possible and further purification steps for the removal of toxicologically unacceptable agents, such as, for example, high concentrations of organic solvents or other toxicologically unacceptable adjuvants, are thus unnecessary before use of the EGFR Fcab-drug conjugates according to the present invention in pharmaceutical formulations.

The invention particularly preferably also relates to pharmaceutical preparations comprising at least one EGFR Fcab-drug conjugate according to the present invention in precipitated non-crystalline, precipitated crystalline or in dissolved or suspended form, and optionally excipients and/or adjuvants and/or further pharmaceutical active compounds.

The EGFR Fcab-drug conjugates according to the present invention preferably enable the preparation of highly concentrated formulations without unfavourable, undesired aggregation of the EGFR Fcab-drug conjugates according to the invention occurring. Thus, ready-to-use solutions having a high active-ingredient content can be prepared with the aid of EGFR Fcab-drug conjugates according to the present invention with aqueous solvents or in aqueous media.

The EGFR Fcab-drug conjugates according to the present invention can also be lyophilised and the resultant lyophilizates used, for example, for the preparation of injection preparations.

Aqueous preparations can be prepared by dissolving or suspending EGFR Fcab-drug conjugates according to the present invention in an aqueous solution and optionally adding adjuvants. To this end, defined volumes of stock solutions comprising the said further adjuvants in defined concentration are advantageously added to a solution or suspension having a defined concentration of EGFR Fcab-drug conjugates according to the present invention, and the mixture is optionally diluted with water to the pre-calculated concentration. Alternatively, the adjuvants can be added in solid form. The amounts of stock solutions and/or water which are necessary in each case can subsequently be added to the aqueous solution or suspension obtained. EGFR Fcab-drug conjugates according to the present invention according to the invention can also advantageously be dissolved or suspended directly in a solution comprising all further adjuvants.

The solutions or suspensions comprising EGFR Fcab-drug conjugates according to the invention and having a pH of 4 to 10, preferably having a pH of 5 to 9, and an osmolality of 250 to 350 mOsmol/kg can advantageously be prepared. The pharmaceutical preparation can thus be administered directly substantially without pain intravenously, intra-arterially, intra-articularly, subcutaneously or percutaneously. In addition, the preparation may also be added to infusion solutions, such as, for example, glucose solution, isotonic saline solution or Ringer's solution, which may also contain further active compounds, thus also enabling relatively large amounts of active compound to be administered.

Pharmaceutical preparations according to the invention may also comprise mixtures of a plurality of EGFR Fcab-drug conjugates according to the present invention.

The preparations according to the invention are physiologically well tolerated, easy to prepare, can be dispensed precisely and are preferably stable with respect to assay, decomposition products and aggregates throughout storage and transport and during multiple freezing and thawing processes. They can preferably be stored in a stable manner over a period of at least three months to two years at refrigerator temperature (2-8° C.) and at room temperature)(23-27° ° C. and 60% relative atmospheric humidity (R.H.).

For example, the EGFR Fcab-drug conjugates according to the present invention can be stored in a stable manner by drying and when necessary converted into a ready-to-use pharmaceutical preparation by dissolution or suspension. Possible drying methods are, for example, without being restricted to these examples, nitro-gen-gas drying, vacuum-oven drying, lyophilisation, washing with organic solvents and subsequent air drying, liquid-bed drying, fluidised-bed drying, spray drying, roller drying, layer drying, air drying at room temperature and further methods.

On use of preparations or medicaments according to the invention, the EGFR Fcab-drug conjugates according to the present invention are generally used analogously to known, commercially available preparations or preparations, preferably in dosages of between 0.1 and 500 mg, in particular 5 and 300 mg, per use unit. The daily dose is preferably between 0.001 and 250 mg/kg, in particular 0.01 and 100 mg/kg, of body weight. The preparation can be administered one or more times per day, for example two, three or four times per day. However, the individual dose for a patient depends on a large number of individual factors, such as, for example, on the efficacy of the particular compound used, on the age, body weight, general state of health, sex, nutrition, on the time and method of administration, on the excretion rate, on the combination with other medicaments and on the severity and duration of the particular disease.

A measure of the uptake of a medicament active compound in an organism is its bioavailability. If the medicament active compound is delivered to the organism intravenously in the form of an injection solution, its absolute bioavailability, i.e. the proportion of the pharmaceutical which reaches the systemic blood, i.e. the major circulation, in unchanged form, is 100%. In the case of oral administration of a therapeutic active compound, the active compound is generally in the form of a solid in the formulation and must therefore first be dissolved in order that it is able to overcome the entry barriers, for example the gastrointestinal tract, the oral mucous membrane, nasal membranes or the skin, in particular the stratum corneum, or can be absorbed by the body. Data on the pharmacokinetics, i.e. on the bioavailability, can be obtained analogously to the method of J. Shaffer et al., J. Pharm. Sciences, 88 (1999), 313-318.

Furthermore, medicaments of this type can be prepared by means of one of the processes generally known in the pharmaceutical art.

Medicaments can be adapted for administration via any desired suitable route, for example by the oral (including buccal or sublingual), rectal, pulmonary, nasal, topical (including buccal, sublingual or transdermal), vaginal or parenteral (including subcutaneous, intramuscular, intravenous, intradermal and in particular intra-articular) routes. Medicaments of this type can be prepared by means of all processes known in the pharmaceutical art by, for example, combining the active EGFR Fcab-drug conjugate with the excipient(s) or adjuvant(s).

Parenteral administration is preferably suitable for administration of the medicaments according to the invention. In the case of parenteral administration, intra-articular administration is particularly preferred.

The EGFR Fcab-drug conjugates according to the invention are also suitable for the preparation of medicaments to be administered parenterally having slow, sustained and/or controlled release of active compound. They are thus also suitable for the preparation of delayed-release formulations, which are advantageous for the patient since administration is only necessary at relatively large time intervals.

The medicaments adapted to parenteral administration include aqueous and non-aqueous sterile injection solutions comprising antioxidants, buffers, bacteriostatics and solutes, by means of which the formulation is rendered isotonic with the blood or synovial fluid of the recipient to be treated; as well as aqueous and non-aqueous sterile suspensions, which can comprise suspension media and thickeners. The formulations can be delivered in single-dose or multi-dose containers, for example sealed ampoules and vials, and stored in the freeze-dried (lyophilised) state, so that only the addition of the sterile carrier liquid, for example water for injection purposes, immediately before use is necessary. Injection solutions and suspensions prepared in accordance with the formulation can be prepared from sterile powders, granules and tablets.

The EGFR Fcab-drug conjugates according to the invention can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The EGFR Fcab-drug conjugates according to the invention can also be coupled to soluble polymers as targeted medicament excipients. Such polymers can encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The EGFR Fcab-drug conjugates according to the invention can furthermore be coupled to a class of biodegradable polymers which are suitable for achieving slow release of a medicament, for example polylactic acid, poly-epsilon-caprolactone, polyhydroxybutyric acid, polyorthoesters, polyacetals, polydihydroxypyrans, polycyanoacrylates, polylactic-co-glycolic acid, polymers, such as conjugates between dextran and methacrylates, polyphosphoesters, various polysaccharides and polyamines and poly-E-caprolactone, albumin, chitosan, collagen or modified gelatine and crosslinked or amphipathic block copolymers of hydrogels.

Suitable for enteral administration (oral or rectal) are, in particular, tablets, dragees, capsules, syrups, juices, drops or suppositories, and suitable for topical use are ointments, creams, pastes, lotions, gels, sprays, foams, aerosols, solutions (for example solutions in alcohols, such as ethanol or isopropanol, acetonitrile, DMF, dimethylacetamide, 1,2-propanediol or mixtures thereof with one another and/or with water) or powders. Also particularly suitable for topical uses are liposomal preparations.

In the case of formulation to give an ointment, the active compound can be employed either with a paraffinic or a water-miscible cream base. Alternatively, the active EGFR Fcab-drug conjugate can be formulated to a cream with an oil-in-water cream base or a water-in-oil base.

Medicaments adapted to transdermal administration can be delivered as independent plasters for extended, close contact with the epidermis of the recipient. Thus, for example, the active EGFR Fcab-drug conjugate can be supplied from the plaster by means of iontophoresis, as described in general terms in Pharmaceutical Research, 3 (6), 318 (1986).

It goes without saying that, besides the constituents particularly mentioned above, the medicaments according to the invention may also comprise other agents usual in the art with respect to the particular type of pharmaceutical formulation.

The EGFR Fcab-drug conjugate described herein may also be in the form of pharmaceutical formulations, pharmaceutical preparations, sets or kits.

The present invention further relates to a set (kit) consisting of separate packs of
a) an effective amount of comprising at least one EGFR Fcab-drug conjugate according to the present invention, and
b) an effective amount of a further medicament active compound.

The set comprises suitable containers, such as boxes or cartons, individual bottles, bags or ampoules. The set may, for example, comprise separate ampoules each containing an effective amount of an EGFR Fcab-drug conjugate according to the present invention and an effective amount of a further medicament active compound in dissolved or lyophilised form.

In one embodiment, the EGFR Fcab-drug conjugate according to the present invention is administered once every 2-6 weeks (e.g. 2, 3 or 4 weeks, in particular 3 weeks). In one embodiment, the EGFR Fcab-drug conjugate is administered for once every two weeks (“Q2W”). In one embodiment, the EGFR Fcab-drug conjugate is administered for once every three weeks (“Q3W”). In one embodiment, the EGFR Fcab-drug conjugate is administered for once every 6 weeks (“Q6W”). In one embodiment, the EGFR Fcab-drug conjugate is administered for Q3W for 2-6 dosing cycles (e.g. the first 3, 4, or 5 dosing cycles, in particular, the first 4 dosing cycles).

In certain embodiments, the cancer to be treated is EGFR positive. For example, in certain embodiments, the cancer to be treated exhibits EGFR+ expression (e.g., high EGFR expression). Methods of detecting a biomarker, such as EGFR for example, on a cancer or tumor, are routine in the art and are contemplated herein. Non-limiting examples include immunohistochemistry, immunofluorescence and fluorescence activated cell sorting (FACS). In some embodiments, subjects or patients with EGFR high cancer are treated by intravenously administering anti-EGFR Fcab-drug conjugate at a dose of about 1200 mg Q2W. In some embodiments, subjects or patients with EGFR high cancer are treated by intravenously administering EGFR Fcab-drug conjugate at a dose of about 1800 mg Q3W. In some embodiments, subjects or patients with EGFR high cancer are treated by intravenously administering EGFR Fcab-drug conjugate at a dose of about 2100 mg Q3W. In some embodiments, subjects or patients with EGFR high cancer are treated by intravenously administering EGFR Fcab-drug conjugate at a dose of about 2400 mg Q3W. In some embodiments, subjects or patients with EGFR high cancer are treated by intravenously administering EGFR Fcab-drug conjugate n at a dose of about 15 mg/kg Q3W.

In certain embodiments, the cancer to be treated has elevated levels of adenosine in the tumor microenvironment.

In certain embodiments, the dosing regimen comprises administering the anti-EGFR Fcab-drug conjugate, at a dose of about 0.01-3000 mg (e.g. a dose about 0.01 mg; a dose about 0.08 mg; a dose about 0.1 mg; a dose about 0.24 mg; a dose about 0.8 mg; a dose about 1 mg; a dose about 2.4 mg; a dose about 8 mg; a dose about 10 mg; a dose about 20 mg; a dose about 24 mg; a dose about 30 mg; a dose about 40 mg; a dose about 48 mg; a dose about 50 mg; a dose about 60 mg; a dose about 70 mg; a dose about 80 mg; a dose about 90 mg; a dose about 100 mg; a dose about 160 mg; a dose about 200 mg; a dose about 240 mg; a dose about 300 mg; a dose about 400 mg; a dose about 500 mg; a dose about 600 mg; a dose about 700 mg; a dose about 800 mg; a dose about 900 mg; a dose about 1000 mg; a dose about 1100 mg; a dose about 1200 mg; a dose about 1300 mg; a dose about 1400 mg; a dose about 1500 mg; a dose about 1600 mg; a dose about 1700 mg; a dose about 1800 mg; a dose about 1900 mg; a dose about 2000 mg; a dose about 2100 mg; a dose about 2200 mg; a dose about 2300 mg; a dose about 2400 mg; a dose about 2500 mg; a dose about 2600 mg; a dose about 2700 mg; a dose about 2800 mg; a dose about 2900 mg; or a dose about 3000 mg). In some embodiments, the dose is a dose of about 500 mg. In some embodiments, the dose is about 1200 mg. In some embodiments, the dose is about 2400 mg. In some embodiments, the dose of the EGFR Fcab-drug conjugate is about 0.001-100 mg/kg (e.g., a dose about 0.001 mg/kg; a dose about 0.003 mg/kg; a dose about 0.01 mg/kg; a dose about 0.03 mg/kg; a dose about 0.1 mg/kg; a dose about 0.3 mg/kg; a dose about 1 mg/kg; a dose about 2 mg/kg; a dose about 3 mg/kg; a dose about 10 mg/kg; a dose about 15 mg/kg; or a dose about 30 mg/kg).

All fixed doses disclosed herein are considered comparable to the body-weight dosing based on a reference body weight of 80 kg. Accordingly, when reference is made to a fixed dose of 2400 mg, a body-weight dose of 30 mg/kg is likewise disclosed therewith.

Concurrent treatment in addition to the treatment with the EGFR Fcab-drug conjugate of the invention and considered necessary for the patient's well-being may be given at discretion of the treating physician. In some embodiments, the present invention provides methods of treating, stabilizing or decreasing the severity or progression of one or more diseases or disorders described herein comprising administering to a patient in need thereof an EGFR Fcab-drug conjugate with an additional therapy, such as chemotherapy, radiotherapy or chemoradiotherapy.

In one embodiment, diterpenoids, such as paclitaxel, nab-paclitaxel or docetaxel; vinca alkaloids, such as vinblastine, vincristine, or vinorelbine; platinum coordination complexes, such as cisplatin or carboplatin; nitrogen mustards such as cyclophosphamide, melphalan, or chlorambucil; alkyl sulfonates such as busulfan; nitrosoureas such as carmustine; triazenes such as dacarbazine; actinomycins such as dactinomycin; anthrocyclins such as daunorubicin or doxorubicin; bleomycins; epipodophyllotoxins such as etoposide or teniposide; antimetabolite anti-neoplastic agents such as fluorouracil, pemetrexed, methotrexate, cytarabine, mecaptopurine, thioguanine, or gemcitabine; methotrexate; camptothecins such as irinotecan or topotecan; rituximab; ofatumumab; trastuzumab; cetuximab; bexarotene; sorafenib; erbB inhibitors such as lapatinib, erlotinib or gefitinib; pertuzumab; ipilimumab; tremelimumab; nivolumab; pembrolizumab; FOLFOX; capecitabine; FOLFIRI; bevacizumab; atezolizumab; selicrelumab; obinotuzumab or any combinations thereof is/are further administered.

In one embodiment, radiotherapy is further administered concurrently or sequentially with the EGFR Fcab-drug conjugate. In some embodiments, the radiotherapy is selected from the group consisting of systemic radiation therapy, external beam radiation therapy, image-guided radiation therapy, tomotherapy, stereotactic radio surgery, stereotactic body radiation therapy, and proton therapy. In some embodiments, the radiotherapy comprises external-beam radiation therapy, internal radiation therapy (brachytherapy), or systemic radiation therapy. See, e.g., Amini et al., Radiat Oncol. “Stereotactic body radiation therapy (SBRT) for lung cancer patients previously treated with conventional radiotherapy: a review” 9:210 (2014); Baker et al., Radiat Oncol. “A critical review of recent developments in radiotherapy for non-small cell lung cancer” 11(1):115 (2016); Ko et al., Clin Cancer Res “The Integration of Radiotherapy with Immunotherapy for the Treatment of Non-Small Cell Lung Cancer” (24) (23) 5792-5806; and, Yamoah et al., Int J Radiat Oncol Biol Phys “Radiotherapy Intensification for Solid Tumors: A Systematic Review of Randomized Trials” 93(4): 737-745 (2015).

In some embodiments, the radiotherapy comprises external-beam radiation therapy, and the external bean radiation therapy comprises intensity-modulated radiation therapy (IMRT), image-guided radiation therapy (IGRT), tomotherapy, stereotactic radiosurgery, stereotactic body radiation therapy, proton therapy, or other charged particle beams.

In some embodiments, the radiotherapy comprises stereotactic body radiation therapy.

Besides the EGFR Fcab-drug conjugate according to the invention, the pharmaceutical preparations according to the invention may also comprise further medicament active compounds, for example for use in the treatment of cancer, other anti-tumor medicaments. For the treatment of the other diseases mentioned, the pharmaceutical preparations according to the invention may also, besides the EGFR Fcab-drug conjugate according to the invention, comprise further medicament active compounds which are known to the person skilled in the art in the treatment thereof.

In one embodiment, the method comprises administering an EGFR Fcab-drug conjugate of the present invention to a host in combination or alternation with an antibody. In particular subembodiments, the antibody is a therapeutic antibody. In one particular embodiment, a method of enhancing efficacy of passive antibody therapy is provided comprising administering an EGFR Fcab-drug conjugate of the present invention in combination or alternation with one or more passive antibodies. This method can enhance the efficacy of antibody therapy for treatment of abnormal cell proliferative disorders such as cancer or can enhance the efficacy of therapy in the treatment or prevention of infectious diseases. The EGFR Fcab-drug conjugate of the present invention can be administered in combination or alternation with antibodies such as rituximab, herceptin or erbitux, for example.

In another principal embodiment, a method of treating or preventing abnormal cell proliferation is provided comprising administering an EGFR Fcab-drug conjugate of the present invention to a host in need thereof substantially in the absence of another anti-cancer agent.

In another principal embodiment, a method of treating or preventing abnormal cell proliferation in a host in need thereof is provided, comprising administering a first an EGFR Fcab-drug conjugate of the present invention substantially in combination with a first anti-cancer agent to the host and subsequently administering a second EGFR Fcab-drug conjugate. In one subembodiment, the second EGFR Fcab-drug conjugate is administered substantially in the absence of another anti-cancer agent. In another principal embodiment, a method of treating or preventing abnormal cell proliferation in a host in need thereof is provided, comprising administering an EGFR Fcab-drug conjugate of the present invention substantially in combination with a first anti-cancer agent to the host and subsequently administering a second anti-cancer agent in the absence of the EGFR Fcab-drug conjugate.

Thus, the cancer treatment disclosed here can be carried out as therapy with an EGFR Fcab-drug conjugate of the present invention or in combination with an operation, irradiation or chemotherapy. Chemotherapy of this type can include the use of one or more active compounds of the following categories of antitumour active compounds:
(i) antiproliferative/antineoplastic/DNA-damaging active compounds and combinations thereof, as used in medical oncology, such as alkylating active compounds (for example cis-platin, parboplatin, cyclophosphamide, nitrogen mustard, melphalan, chlorambucil, busulphan and nitrosoureas); antimetabolites (for example antifolates such as fluoropyrimidines such as 5-fluorouracil and tegafur, raltitrexed, methotrexate, cytosine arabinoside, hydroxyurea and gemcitabine); antitumor antibiotics (for example anthracyclines, such as adriamycin, bleomycin, doxorubicin, daunomycin, epirubicin, idarubicin, mitomycin-C, dactinomycin and mithramycin); antimitotic active compounds (for example vinca alkaloids, such as vincristine, vinblastine, vindesine and vinorelbine, and taxoids, such as taxol and taxotere); topoisomerase inhibitors (for example epipodophyllotoxins, such as etoposide and teniposide, amsacrine, topotecan, irinotecan and camptothecin) and cell-differentiating active compounds (for example all-trans-retinoic acid, 13-cis-retinoic acid and fenretinide);
(ii) cytostatic active compounds, such as anti-oestrogens (for example tamoxifen, toremifene, raloxifene, droloxifene and iodoxyfene), oestrogen receptor regulators (for example fulvestrant), anti-androgens (for example bicalutamide, flutamide, nilutamide and cyproterone acetate), LHRH antagonists or LHRH agonists (for example goserelin, leuprorelin and buserelin), progesterones (for example megestrol acetate), aromatase inhibitors (for example anastrozole, letrozole, vorazole and exemestane) and inhibitors of 5×-reductase, such as finasteride;
(iii) active compounds which inhibit cancer invasion including for example metallo-proteinase inhibitors, like marimastat, and inhibitors of urokinase plasminogen activator receptor function;
(iv) inhibitors of growth factor function, for example growth factor antibodies, growth factor receptor antibodies, for example the anti-erbb2 antibody trastuzumab [Herceptin™] and the anti-erbbl antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors, such as N-(3-chloro-4-fluorophenyl)-7-methoxy-6-(3-morpholinopropoxy) quinazolin-4-amine (gefitinib, AZD1839), N-(3-ethynylphenyl)-6,7-bis (2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033), for example inhibitors of the platelet-derived growth factor family and, for example, inhibitors of the hepatocyte growth factor family;
(v) anti-angiogenic active compounds, such as bevacizumab, angiostatin, endostatin, linomide, batimastat, captopril, cartilage derived inhibitor, genistein, interleukin 12, lavendustin, medroxypregesterone acetate, recombinant human platelet factor 4, tecogalan, thrombospondin, TNP-470, anti-VEGF monoclonal antibody, soluble VEGF-receptor chimaeric protein, anti-VEGF receptor antibodies, anti-PDGF receptors, inhibitors of integrins, tyrosine kinase inhibitors, serine/threonine kinase inhibitors, antisense oligonucleotides, antisense oligodexoynucleotides, siRNAs, anti-VEGF aptamers, pigment epithelium derived factor and compounds which have been published in the international patent applications WO 97/22596, WO 97/30035, WO 97/32856 and WO 98/13354);
(vi) vessel-destroying agents, such as combretastatin A4 and compounds which have been published in the international patent applications WO 99/02166, WO 00/40529, WO 00/41669, WO 01/92224, WO 02/04434 and WO 02/08213;
(vii) antisense therapies, for example those directed to the targets mentioned above, such as ISIS 2503, an anti-Ras antisense;
(viii) gene therapy approaches, including, for example, approaches for replacement of abnormal, modified genes, such as abnormal p53 or abnormal BRCA1 or BRCA2, GDEPT approaches (gene-directed enzyme pro-drug therapy), such as those which use cytosine deaminase, thymidine kinase or a bacterial nitroreductase enzyme, and approaches which increase the tolerance of a patient to chemotherapy or radiotherapy, such as multi-drug resistance therapy; and
(ix) immunotherapy approaches, including, for example, ex-vivo and in-vivo approaches for increasing the immunogenicity of tumor cells of a patient, such as transfection with cytokines, such as interleukin 2, interleukin 4 or granulocyte macrophage colony stimulating factor, approaches for decreasing T-cell anergy, approaches using transfected immune cells, such as cytokine-transfected dendritic cells, approaches for use of cytokine-transfected tumor cells and approaches for use of anti-idiotypic antibodies

(x) chemotherapeutic agents including for example abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, epirubicin, epoetin alfa, estramustine, etoposide, exemestane, filgrastim, floxuridine, fludarabine, fluorouracil, fulvestrant and gemcitabine.

The medicaments from table 1 can preferably, but not exclusively, be combined with the EGFR Fcab-drug conjugates of the present invention.

TABLE 1

| | | |
|---|---|---|
| Alkylating active | Cyclophosphamide | Lomustine |
| compounds | Busulfan | Procarbazine |
| | Ifosfamide | Altretamine |
| | Melphalan | Estramustine phosphate |
| | Hexamethylmelamine | Mechloroethamine |
| | Thiotepa | Streptozocin |
| | chloroambucil | Temozolomide |
| | Dacarbazine | Semustine |
| | Carmustine | |
| Platinum active | Cisplatin | Carboplatin |
| compounds | Oxaliplatin | ZD-0473 (AnorMED) |
| | Spiroplatin | Lobaplatin (Aetema) |
| | Carboxyphthalatoplatinum | Satraplatin (Johnson |
| | Tetraplatin | Matthey) |
| | Ormiplatin | BBR-3464 |
| | Iproplatin | (Hoffrnann-La Roche) |
| | | SM-11355 (Sumitomo) |
| | | AP-5280 (Access) |
| Antimetabolites | Azacytidine | Tomudex |
| | Gemcitabine | Trimetrexate |
| | Capecitabine | Deoxycoformycin |
| | 5-Fluorouracil | Fludarabine |
| | Floxuridine | Pentostatin |
| | 2-Chlorodesoxyadenosine | Raltitrexed |
| | 6-Mercaptopurine | Hydroxyurea |
| | 6-Thioguanine | Decitabine (SuperGen) |
| | Cytarabine | Clofarabine (Bioenvision) |
| | 2-Fluorodesoxycytidine | Irofulven (MGI Pharrna) |
| | Methotrexate | DMDC (Hoffmann-La Roche) |
| | Idatrexate | Ethynylcytidine (Taiho) |
| Topoisomerase | Amsacrine | Rubitecan (SuperGen) |
| inhibitors | Epirubicin | Exatecan mesylate (Daiichi) |
| | Etoposide | Quinamed (ChemGenex) |
| | Teniposide or mitoxantrone | Gimatecan (Sigma- Tau) |
| | Irinotecan (CPT-11) | Diflomotecan (Beaufour- |
| | 7-ethyl-10- | Ipsen) |
| | hydroxycamptothecin | TAS-103 (Taiho) |
| | Topotecan | Elsamitrucin (Spectrum) |
| | Dexrazoxanet (TopoTarget) | J-107088 (Merck & Co) |
| | Pixantrone (Novuspharrna) | BNP-1350 (BioNumerik) |
| | Rebeccamycin analogue | CKD-602 (Chong Kun Dang) |
| | (Exelixis) | KW-2170 (Kyowa Hakko) |
| | BBR-3576 (Novuspharrna) | |
| Antitumour | Dactinomycin (Actinomycin D) | Amonafide |
| antibiotics | Doxorubicin (Adriamycin) | Azonafide |
| | Deoxyrubicin | Anthrapyrazole |
| | Valrubicin | Oxantrazole |
| | Daunorubicin (Daunomycin) | Losoxantrone |
| | Epirubicin | Bleomycin sulfate (Blenoxan) |
| | Therarubicin | Bleomycinic acid |
| | Idarubicin | Bleomycin A |
| | Rubidazon | Bleomycin B |
| | Plicamycinp | Mitomycin C |
| | Porfiromycin | MEN-10755 (Menarini) |
| | Cyanomorpholinodoxorubicin | GPX-100 (Gem |
| | Mitoxantron (Novantron) | Pharmaceuticals) |
| Antimitotic active | Paclitaxel | SB 408075 |
| compounds | Docetaxel | (GlaxoSmithKline) |
| | Colchicine | E7010 (Abbott) |
| | Vinblastine | PG-TXL (Cell Therapeutics) |
| | Vincristine | IDN 5109 (Bayer) |
| | Vinorelbine | A 105972 (Abbott) |
| | Vindesine | A 204197 (Abbott) |
| | Dolastatin 10 (NCI) | LU 223651 (BASF) |
| | Rhizoxin (Fujisawa) | D 24851 (ASTA Medica) |
| | Mivobulin (Warner-Lambert) | ER-86526 (Eisai) |
| | Cemadotin (BASF) | Combretastatin A4 (BMS) |
| | RPR 109881A (Aventis) | Isohomohalichondrin-B |
| | TXD 258 (Aventis) | (PharmaMar) |
| | Epothilone B (Novartis) | ZD 6126 (AstraZeneca) |
| | T 900607 (Tularik) | PEG-Paclitaxel (Enzon) |
| | T 138067 (Tularik) | AZ10992 (Asahi) |
| | Cryptophycin 52 (Eli Lilly) | IDN-5109 (Indena) |
| | Vinflunine (Fabre) | AVLB (Prescient |
| | Auristatin PE (Teikoku | NeuroPharma) |
| | Hormone) | Azaepothilon B (BMS) |
| | BMS 247550 (BMS) | BNP- 7787 (BioNumerik) |
| | BMS 184476 (BMS) | CA-4-prodrug (OXiGENE) |
| | BMS 188797 (BMS) | Dolastatin-10 (NrH) |
| | Taxoprexin (Protarga) | CA-4 (OXiGENE) |
| Aromatase | Aminoglutethimide | Exemestan |
| inhibitors | Letrozole | Atamestan (BioMedicines) |
| | Anastrazole | YM-511 (Yamanouchi) |
| | Formestan | |
| Thymidylate | Pemetrexed (Eli Lilly) | Nolatrexed (Eximias) |
| Synthase | ZD-9331 (BTG) | CoFactor ™ (BioKeys) |
| inhibitors | | |
| DNA antagonists | Trabectedin (PharmaMar) | Mafosfamide (Baxter |
| | Glufosfamide (Baxter | International) |
| | International) | Apaziquone (Spectrum |
| | Albumin + 32P | Pharmaceuticals) |
| | (isotope solutions) | O6-benzylguanine (Paligent) |
| | Thymectacin (NewBiotics) | |
| | Edotreotid (Novartis) | |
| Farnesyl transferase | Arglabin (NuOncology Labs) | Tipifarnib (Johnson & |
| inhibitors | Lonafarnib (Schering-Plough) | Johnson) |
| | BAY-43-9006 (Bayer) | Perillyl alcohol (DOR |
| | | BioPharma) |
| Pump inhibitors | CBT-1 (CBA Pharma) | Zosuquidar trihydrochloride |
| | Tariquidar (Xenova) | (Eli Lilly) |
| | MS-209 (Schering AG) | Biricodar dicitrate (Vertex) |
| Histone acetyl trans- | Tacedinaline (Pfizer) | Pivaloyloxymethyl butyrate |
| ferase inhibitors | SAHA (Aton Pharma) | (Titan) |
| | MS-275 (Schering AG) | Depsipeptide (Fujisawa) |
| Metalloproteinase | Neovastat (Aeterna | CMT -3 (CollaGenex) |
| inhibitors | Laboratories) | BMS-275291 (Celltech) |
| Ribonucleoside | Marimastat (British Biotech) | Tezacitabine (Aventis) |
| reductase | Gallium maltolate (Titan) | Didox (Molecules for Health) |
| inhibitors | Triapin (Vion) | |
| TNF-alpha | Virulizin (Lorus Therapeutics) | Revimid (Celgene) |
| agonists/ | CDC-394 (Celgene) | |
| antagonists | | |
| Endothelin-A re- | Atrasentan (Abbot) | YM-598 (Yamanouchi) |
| ceptor antagonists | ZD-4054 (AstraZeneca) | |
| Retinoic acid | Fenretinide (Johnson & | Alitretinoin (Ligand) |
| receptor agonists | Johnson) | |
| | LGD-1550 (ligand) | |
| Immunomodulators | Interferon | Dexosome therapy (Anosys) |
| | Oncophage (Antigenics) | Pentrix (Australian Cancer |
| | GMK (Progenics) | Technology) |
| | Adenocarcinoma vaccine | JSF-154 (Tragen) |
| | (Biomira) | Cancer vaccine (Intercell) |
| | CTP-37 (AVI BioPharma) | Norelin (Biostar) |
| | JRX-2 (Immuno-Rx) | BLP-25 (Biomira) |
| | PEP-005 (Peplin Biotech) | MGV (Progenics) |
| | Synchrovax vaccines (CTL | !3-Alethin (Dovetail) |
| | Immuno) | CLL-Thera (Vasogen) |
| | Melanoma vaccines (CTL | |
| | Immuno) | |
| | p21-RAS vaccine (GemVax) | |
| Hormonal and | Oestrogens | Prednisone |
| antihormonal active | Conjugated oestrogens | Methylprednisolone |
| compounds | Ethynyloestradiol | Prednisolone |
| | Chlorotrianisene | Aminoglutethimide |
| | Idenestrol | Leuprolide |
| | Hydroxyprogesterone | Goserelin |
| | caproate | Leuporelin |
| | Medroxyprogesterone | Bicalutamide |
| | Testosterone | Flutamide |
| | Testosterone propionate | Octreotide |
| | Fluoxymesterone | Nilutamide |
| | Methyltestosterone | Mitotan |
| | Diethylstilbestrol | P-04 (Novogen) |
| | Megestrol | 2-Methoxyoestradiol (En _- |
| | Tamoxifen | treMed) |
| | Toremofin | Arzoxifen (Eli Lilly) |
| | Dexamethasone | |
| Photodynamic | Talaporfin (Light Sciences) | Pd bacteriopheophorbide |
| active compounds | Theralux (Theratechnologies) | (Yeda) |
| | Motexafin-Gadolinium | Lutetium texaphyrin |
| | (Pharmacyclics) | (Pharmacyclics) |
| | | Hypericin |
| Tyrosine kinase | Imatinib (Novartis) Leflunomide(Sugen/Pharmacia] | CEP- 701 (Cephalon) |
| inhibitors | ZDI839 (AstraZeneca) | Kahalide F (PharmaMar) |
| | Erlotinib (Oncogene Science) | CEP-751 (Cephalon) |
| | Canertjnib (Pfizer) | MLN518 (Millenium) |
| | Squalamine (Genaera) | PKC412 (Novartis) |
| | SU5416 (Pharmacia) | Phenoxodiol O |
| | SU6668 (Pharmacia) | Trastuzumab (Genentech) |
| | ZD4190 (AstraZeneca) | C225 (ImClone) |
| | ZD6474 (AstraZeneca) | rhu-Mab (Genentech) |
| | Vatalanib (Novartis) | MDX-H210 (Medarex) |
| | PKI166 (Novartis) | 2C4 (Genentech) |
| | GW2016 (GlaxoSmithKline) | MDX-447 (Medarex) |
| | EKB-509 (Wyeth) | ABX-EGF (Abgenix) |
| | EKB-569 (Wyeth) | IMC-1C11 (ImClone) |
| Various other active | SR-27897 (CCK-A inhibitor, | BCX-1777 (PNP inhibitor, |
| compounds | Sanofi-Synthelabo) | BioCryst) |
| | Tocladesine (cyclic AMP | Ranpirnase (ribonuclease |
| | agonist, Ribapharm) | stimulant, Alfacell) |
| | Alvocidib (CDK inhibitor, | Galarubicin (RNA synthesis |
| | Aventis) | inhibitor, Dong-A) |
| | CV-247 (COX-2 inhibitor, Ivy | Tirapazamine (reducing |
| | Medical) | agent, SRI International) |
| | P54 (COX-2 inhibitor, | N-Acetylcysteine |
| | Phytopharm) | (reducing agent, |
| | CapCell ™ (CYP450 | Zambon) |
| | stimulant, Bavarian Nordic) | R-Flurbiprofen (NF-kappaB |
| | GCS-IOO (gal3 antagonist, | inhibitor, Encore) |
| | GlycoGenesys) | 3CPA (NF-kappaB inhibitor, |
| | G17DT immunogen (gastrin | Active Biotech) |
| | inhibitor, Aphton) | Seocalcitol (vitamin D |
| | Efaproxiral (oxygenator, | receptor agonist, Leo) |
| | Allos Therapeutics) | 131-I-TM-601 (DNA |
| | PI-88 (heparanase inhibitor, | antagonist, TransMolecular) |
| | Progen) | Eflornithin (ODC inhibitor, |
| | Tesmilifen (histamine | ILEX Oncology) |
| | antagonist, YM BioSciences) | Minodronic acid (osteoclast |
| | Histamine (histamine H2 | inhibitor, |
| | receptor agonist, Maxim) | Yamanouchi) |
| | Tiazofurin (IMPDH inhibitor, | Indisulam (p53 stimulant, |
| | Ribapharm) | Eisai) |
| | Cilengitide (integrin antagonist, | Aplidin (PPT inhibitor, |
| | Merck KGaA) | PharmaMar) |
| | SR-31747 (IL-1 antagonist, | Rituximab (CD20 antibody, |
| | Sanofi-Synthelabo) | Genentech) |
| | CCI-779 (mTOR kinase | Gemtuzumab (CD33 |
| | inhibitor, Wyeth) | antibody, Wyeth Ayerst) |
| | Exisulind (PDE-V inhibitor, | PG2 (haematopoiesis |
| | Cell Pathways) | promoter, Pharmagenesis) |
| | CP-461 (PDE-V inhibitor, Cell | Immunol ™ (triclosan |
| | Pathways) | mouthwash, Endo) |
| | AG-2037 (GART inhibitor, | Triacetyluridine (uridine |
| | Pfizer) | prodrug, Wellstat) |
| | WX-UK1 (plasminogen | SN-4071 (sarcoma agent, |
| | activator inhibitor, Wilex) | Signature BioScience) |
| | PBI-1402 (PMN stimulant, | TransMID-107 ™ |
| | ProMetic LifeSciences) | (immunotoxin, KS Biomedix) |
| | Bortezomib (proteasome | PCK-3145 (apoptosis pro- |
| | inhibitor, Millennium) | moter, Procyon) |
| | SRL-172 (T-cell stimulant, | Doranidazole (apoptosis pro- |
| | SR Pharma) | moter, Pola) |
| | TLK-286 (glutathione-S | CHS-828 (cytotoxic agent, |
| | transferase inhibitor, Telik) | Leo) |
| | PT-100 (growth factor | trans-Retinoic acid ( |
| | agonist, Point Therapeutics) | differentiator, NIH) |
| | Midostaurin (PKC inhibitor, | MX6 (apoptosis promoter, |
| | Novartis) | MAXIA) |
| | Bryostatin-1 (PKC stimulant, | Apomine (apoptosis |
| | GPC Biotech) | promoter, ILEX Oncology) |
| | CDA-II (apoptosis promoter, | Urocidin (apoptosis promoter, |
| | Everlife) | Bioniche) |
| | SDX-101 (apoptosis promoter, | Ro-31-7453 (apoptosis pro- |
| | Salmedix) | moter, La Roche) |
| | Ceflatonin (apoptosis pro- | Brostallicin (apoptosis |
| | moter, ChemGenex) | promoter, Pharmacia) |

The disclosure further provides diagnostic, predictive, prognostic and/or therapeutic methods using the EGFR Fcab-dyeg conjugate described herein. Such methods are based, at least in part, on determination of the identity of the expression level of a biomarker of interest. In particular, the amount of any one of human EGFR in a cancer patient sample can be used as a biomarker to predict whether the patient is likely to respond favorably to cancer therapy utilizing the therapeutic combination of the invention.

Thus, another embodiment of the present invention is an EGFR Fcab-label conjugate comprising the formula Fcab-(L)ₘ-(La)ₙ wherein:
a) Fcab comprises an EGFR Fcab,
b) L comprises a linker,
c) La comprises a label,
d) m is an integer from 1-5 and n is an integer from 1-10.

A preferred embodiment of the present invention is an EGFR Fcab-label conjugate according to the present invention wherein the EGFR Fcab is selected from the group consisting of: Fcab-1, Fcab-2, Fcab-3, Fcab-4, Fcab-5 and Fcab-6, having the amino acid sequences as set forth in SEQ ID Nos. 1-6.

A further preferred embodiment of the present invention is an EGFR Fcab-label conjugate according to the present invention wherein the EGFR Fcab is selected from the group consisting of: Fcab-1, Fcab-2 and Fcab-3, having the amino acid sequences as set forth in SEQ ID Nos. 1-3.

In a preferred embodiment of the present invention m is 1 to 3 and n is 1 to 5.

The invention relates also to EGFR Fcab-label conjugates in which the EGFR Fcab according to the present invention are modified by adding a label, yielding labelled EGFR Fcab conjugates. The label can be coupled to the EGFR Fcab via spacers/linkers of various lengths to reduce potential steric hindrance. The linkers can be the same as described above for the EGFR Fcab-drug conjugates according to the present invention.

The term “label” or “labelling group” refers to any detectable label. Exemplary labels include, but are not limited to isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I); magnetic labels (e.g., magnetic particles); redox active moieties; optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluorophores or proteinaceous fluorophores; enzymatic groups (e.g., horseradish peroxidase, ˜-galactosidase, luciferase, alkaline phosphatase; biotinylated groups; or predetermined polypeptide epitopes recognized by a secondary reporter (e.g., leucine zipper pair sequences, binding sites for secondary antibodies, metal binding domains, epitope tags, etc.).

A preferred embodiment of the present invention is an EGFR Fcab-label conjugate of the present invention wherein the label is selected from the group consisting of an isotopic label, a magnetic label, a redox active moiety, an optical dye and an enzymatic group.

A further preferred embodiment of the present invention is an EGFR Fcab-label conjugate of the present invention wherein the label is a pHAb-dye.

A label according to the present invention can also be a tag, such as an affinity tag aiding in purification and isolation of the antibody. Non-limiting examples of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof(e.g. StrepII-tag) and His-tag.

Thus, a further preferred embodiment of the present invention is an EGFR Fcab-label conjugate of the present invention wherein the label is a tag.

Another embodiment of the present invention is a diagnostic composition containing the EGFR Fcab-label conjugates according to the present invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the biomarker protein or other suitable read-outs for biomarker levels, respectively, examples of which are described herein and/or are well known to the skilled artisan.

In some embodiments, determining the biomarker level comprises determining the biomarker expression. In some embodiments, the biomarker level is determined by the biomarker protein concentration in a patient sample, e.g., with biomarker specific ligands, such as antibodies or specific binding partners. The binding event can, e.g., be detected by competitive or non-competitive methods, including the use of a labeled ligand or biomarker specific moieties, e.g., antibodies, or labeled competitive moieties, including a labeled biomarker standard, which compete with labeled proteins for the binding event. If the biomarker specific ligand is capable of forming a complex with the biomarker, the complex formation can indicate biomarker expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In one embodiment, the biomarker expression is determined by immunohistochemistry using one or more primary antibodies that specifically bind the biomarker.

However, in a preferred embodiment of the present invention the EGFR Fcab-label conjugate according to the present invention is used to determine the expression of EGFR protein in cells, organoids, serum sample, plasma sample, whole blood, pancreatic juice sample, tissue sample, tumor lysate or a tumor sample.

In one embodiment, the efficacy of the therapeutic combination of the invention is predicted by means of EGFR expression in tumor samples.

This disclosure also provides a kit for determining if the combination of the invention is suitable for therapeutic treatment of a cancer patient, comprising means for determining a protein level of EGFR, in a sample isolated from the patient and instructions for use In one aspect of the invention, the determination of a high EGFR level indicates increased PFS or OS when the patient is treated with the EGFR Fcab-drug conjugate of the invention. In one embodiment of the kit, the means for determining the biomarker protein level are antibodies with specific binding to the biomarker.

### BRIEF DESCRIPTION OF THE FIGURES

FIG. 1 shows a conceptual representation of the advantages of Fcab-drug conjugates over other antibody-fragment based drug conjugates (VHH11.12, scFv8.9, Fab6.7) and conventional IgG-based ADCs4.

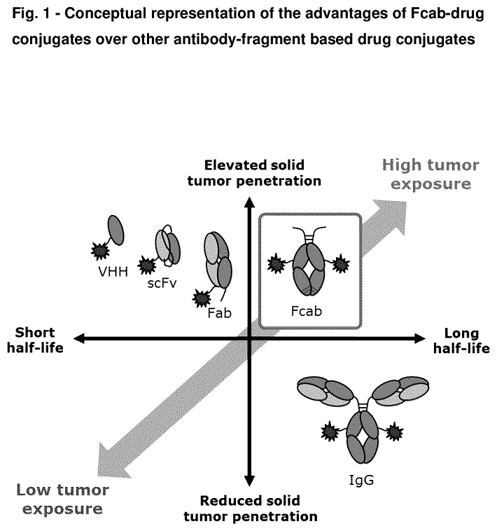

FIG. 2 shows a graphical abstract of the EGFR Fcab-drug conjugates of the present invention.

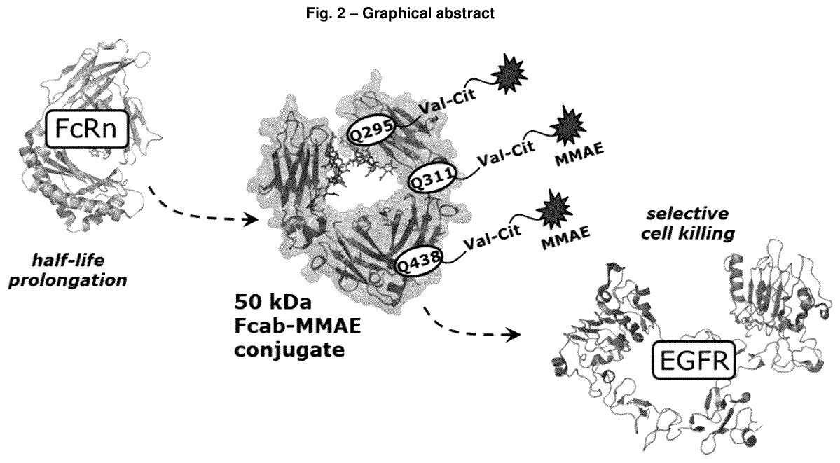

FIG. 3 shows the Fcab structure and pHAb-dye labeling. (A) Schematic representation of homodimeric Fcab with engineered antigen binding site at the C-terminus of the C_{H}3 domain. (B) Human IgG1-Fc monomer depicting the C_{H}3 AB, CD and EF loops that were engineered in the selected EGFR-binding Fcabs. Glycosylation is not shown for clarity. (PDB ID 5JII). (C) Schematic representation of pHAb-dye labeling.

FIG. 4 shows the intracellular accumulation of Fcab-, huFc-, C-Fab- and C-IgG-pHAb dye conjugates relative to the accumulation rate of C-IgG-pHAb on MDA-MB-468 cells (100%). Intracellular accumulation rates were derived from 0-26 h incubation at 100 nM and normalized to cell number and individual pHAb-dye fluorescence of each construct. Relative EGFR expression profiles (MDA-MB-468>A431>MCF-7) are indicated below bar graph. Error bars show the standard deviation of triplicates.

FIG. 5 shows Fcab-drug conjugates. (A) Representative figure of a human Fc portion (PDB ID 5VGP) showing the EGFR binding site located in the C_{H}3 region as well as the conjugation sites Q295, Q311 and Q438 (EU numbering). (B) Structure of linker-payload 1. (C) Schematic representation of MMAE conjugation.

FIG. 6 shows the conjugation site identification. LC-MS chromatogram of digested Fcab-1-MMAE shows conjugated peptides (e-i, k-m) that were not detected in the Fcab-1 preparation. Conjugated peptides eluted at higher retention times due to hydrophobic MMAE. Matched pairs of unconjugated peptides (a-d) eluted at lower retention times and peaks disappeared (a,b) or showed reduced intensity (c) in the Fcab-1-MMAE preparation compared to Fcab-1.

FIG. 7 shows the cell proliferation assays. (A) Cell viability on EGFR positive (MDA-MB-468, A431) and EGFR negative cells (MCF-7). Cells were incubated with serial dilution of MMAE conjugates and free MMAE for 4 days before cell viability was analyzed. Error bars represent standard deviation (SD) of triplicates (B) Inhibitory activity of MMAE conjugates and free MMAE. IC₅₀ values are given as mean of three independent experiments (IC₅₀±SD).

FIG. 8 shows the Protein A purification process of expressed Fcabs (A) Exemplary ÄKTA Xpress (HiTrap™ MabSelect SuRe™ 5 mL and HiPrep™ 26/10 desalting column) chromatogram showing Fcab-2 protein peak after elution from protein A column (50 mM acetic acid (HOAc), pH 3.2) and a second protein peak after a subsequent buffer change step. (B) SDS-PAGE analysis of reduced Expi293F supernatant, protein A flow through and purified Fcabs. 4-12% Bis-Tris gel (Invitrogen™), MES SDS running buffer (1×), 40 min at 200 V, stained with InstantBlue™ (Coomassie-based) for 2h, marker: Precision Plus Protein™ Unstained Standards (BioRad). (C) Yields of purified Fcabs and huFc negative control per volume Expi-293F expression culture.

FIG. 9 shows, that the LC-MS analysis confirms the identity of Fcabs and huFc controls. (A) Deconvoluted MS spectra of Fcabs. (B) Mass variations between calculated and observed masses account for glycosylation patterns and standard measurement deviations. Only the most intense glycosylation pattern (G1F) is listed.

FIG. 10 Thermal stability of Fcabs and huFc control molecule. The first derivative of thermal unfolding curves (A) as well as the unfolding transition midpoints (Tm) (B) are shown. To determine thermal unfolding, Fcabs and huFc (PBS pH 6.8) were loaded into nanoDSF grade standard capillaries which were then transferred into a Prometheus NT.PLEX nanoDSF (NanoTemper Technologies) instrument. Samples were subjected to a linear thermal ramp from 20° C. to 95° C. at a slope of 1° C./min with simultaneous recording of fluorescence at 350 and 330 nm. Unfolding transition midpoints (Tm) were determined from the first derivative of the fluorescence ratio 350 nm/330 nm. All samples were measured in duplicates. n.d.—not detected

FIG. 11 shows the cellular binding analysis of Fcabs and control molecules on EGFR positive (MDA MB 468 and A431) and EGFR negative (MCF 7) cells. Fcabs and C-IgG bind selectively EGFR expressing cells while huFc and secondary detection antibody do not show any binding. Cells were incubated with 100 nM of Fcab/C IgG for 60 min at 4° C., washed twice with PBS-1% BSA, incubated for 30 min with 500 nM of AF488-labeled detection antibody (109-546-008, Jackson ImmunoResearch) at 4° C. in darkness, washed twice with PBS-1% BSA, and finally fluorescence intensity was measured applying an Attune NxT flow cytometer (Invitrogen™).

FIG. 12 shows the determination of cellular dissociation constants (KD). (A) EGFR positive (MDA MB 468 and A431) and EGFR negative (MCF 7) cells were incubated with Fcabs at different concentration followed by staining with AF488-labeled detection antibody, washing and cytometer analysis as described in Figure S4. Varying binding saturation levels between MDA MB 468 and A431 cells reflect distinct cell specific EGFR expression densities. Dose response curves were fitted using the asymmetric (five parameter) fitting function of GraphPad Prism (GraphPad Software, Inc.) to obtain KD. (B) Cellular dissociation constants of Fcabs are in good agreement with KD values derived from binding to recombinant EGFR via BLI (Table 2) and literature data[1].

FIG. 13 shows the pHAb-dye labeling and degree of labeling (DOLF, DOLA) determination (A) Structure of pHAb amine reactive dye 2 carrying an NHS ester group which can react with the E-amino group of lysine. (B) Absorption and fluorescence spectra of pHAb dye in SE-HPLC buffer (pH 6.3). (C) Proteins are labeled with pHAb-dye via random lysine coupling. Several factors can impact fluorophore quantum yield.^{[4]} (D) Overview of the established SE-HPLC method to determine the DOLF and DOLA value. Unconjugated proteins, free pHAb-dye and pHAb-dye conjugates were analyzed by an SE-HPLC device equipped with a diode array (DAD) and a fluorescence (FLD) detector. The molar extinction coefficients (MEC) of unconjugated protein and free pHAb-dye as well as the molar fluorescence coefficient (MFC) of free pHAb-dye were calculated from peak areas and the injected amount of substance. MEC and MFC were then used to calculate the amount of conjugated fluorescent pHAb-dye and the amount of protein from pHAb-dye conjugate peak areas. Finally, DOLF and DOLA can be derived from these data.

FIG. 14 shows the SE-HPLC analysis for the determination of the DOLF and DOLA, exemplarily shown for a pHAb-dye conjugated Fcab. (A) Unconjugated Fcab does not absorb light at 535 nm. (B) Hence, Fcab does not show fluorescence at 566 nm when excited at 535 nm (pHAb-dye absorption maximum). (C) Absorption of Fcab at 280 nm (aromatic amino acids). Fcab (54 kDa) elutes at 9.3 min. (D) Absorption of free pHAb-dye at its absorption maximum at 535 nm. According to its smaller size, free pHAb-dye (786 g/mol, carboxylic acid form) elutes later at 11.2 min. (E) Fluorescence at 566 nm (pHAb-dye fluorescence maximum) of free pHAb-dye when excited at 535 nm. (F) Free pHAb-dye absorbs also at 280 nm. The smaller peak at 11.8 min is caused by buffer components and was neglected for peak integration. (G) Absorption at 535 nm of Fcab pHAb conjugate. The first peak marked in red represents the absorption of conjugated pHAb-dye molecules while the second peak marked in blue shows free pHAb-dye which could not be removed entirely by Zeba™ spin desalting column purification. (H) Fluorescence at 566 nm of Fcab pHAb conjugate. The peak depicted in red shows fluorescence of conjugated pHAb-dye while the blue peak shows fluorescence of free pHAb-dye. When comparing the peak area of fluorescence and absorption peaks (535 nm), fluorescence of conjugated pHAb-dye is much lower with respect to free pHAb-dye. This confirms influences of the local molecular environment on fluorophore quantum yield. (I) Absorption of Fcab pHAb at 280 nm is composed of the absorption of its protein and pHAb-dye components.

FIG. 15 shows the characterization of pHAb-dye conjugates used in this study. (A) Analytical size exclusion SE-HPLC of purified pHAb-dye conjugates showing absorption at 280 nm (protein and pHAb-dye) and 535 nm (pHAb-dye) as well as pHAb-dye fluorescence (Exc. 535 nm, Em. 566 nm). At tR 11-12 min unconjugated pHAb-dye may elute. (B) Absorption- and fluorescence-based degree of pHAb-dye labeling (DOLF, DOLA) derived from SE-HPLC data shown in (A).

FIG. 16 shows the cellular uptake kinetics of pHAb-dye labeled constructs. (A) Cellular accumulation time series exemplarily shown for Fcab 1 pHAb on MDA MB 468 cells. Cells were incubated at 37° C., 80% humidity and 5% CO2 with 100 nM Fcab 1 pHAb and RFP channel images (ex.: 531 nm, em.: 593 nm) were recorded every 2 h for 26 h using a Cytation 5 cell imaging reader (BioTek) equipped with DAPI and RFP filter cubes and a BioSpa 8 automated incubator (BioTek). (B) The total fluorescence intensity of images is normalized to cell-number and DOLF value of the pHAb-dye labeled construct and plotted over time to derive normalized intracellular accumulation rates from slopes of linear regression (GraphPad Software, Inc.). Subsequently, the relative intracellular accumulation can be calculated from these rates. (C) Normalized cellular uptake rates derived from described imager-based assay could be verified in a second FACS-based cellular uptake assay employing the same pHAb-dye labeled constructs. (D) Relative intracellular accumulation in MDA MB 468 derived from FACS-based assay is in good agreement with the imaging-based cellular uptake data (FIG. 3).

FIG. 17 shows the conjugation and purification strategy exemplarily shown for Fcab-MMAE conjugates. (A) MMAE conjugates were generated by enzymatic transglutaminase conjugation. After conjugation, excess of microbial transglutaminase (mTG) and Gly₃-Val-Cit-MMAE (1) was removed by preparative SEC. (B) Purification of transglutaminase conjugated MMAE constructs by preparative SEC, exemplarily shown for Fcab-2-MMAE and huFc-MMAE. Fractions containing conjugated proteins (and non-conjugated species) were pooled, concentrated, sterile filtered and subjected to analytics.

FIG. 18 shows the chromatographic characterization of generated MMAE conjugates exemplarily shown for Fcab-1 MMAE, Fcab-2 MMAE Fcab-3 MMAE and huFc MMAE. (A) Analytical size exclusion SE-HPLC shows a distinct single peak demonstrating formation of monomeric drug conjugates without aggregates. Signal intensity represents absorption at 214 nm (B) Reversed phase RP-HPLC reveals conjugation of Gly3-Val-Cit-MMAE 1. RP-DAR is calculated from peak areas of individual DAR species (Fcab 1 MMAE RP DAR 2.6; Fcab 2 MMAE RP DAR 2.5; Fcab 3 MMAE RP DAR 2.5; huFc MMAE RP DAR 2.0). For example, 25% relative peak area of DAR 1 species and 75% relative peak area of DAR 2 species reveals a final RP-DAR of 1.75. Signal intensity represents absorption at 214 nm.

FIG. 19 shows the DAR determination of Fcab-MMAE conjugates by mass spectrometry. Deconvoluted MS spectra showing groups of differently glycosylated heavy chains (HC) carrying 0-3 Gly3-Val-Cit-MMAE (1). TIC area of HC species carrying 0-3 linker payloads were used to calculate the MS-DAR.

FIG. 20 shows the kinetic parameters EGFR binding. Dissociation constants (KD), on-(kon) and off-rates (koff) were measured at pH 7.4 by BLI using recombinantly produced EGFR. Errors are standard errors from fitting using ForteBio data analysis software 9.1. Fitting quality is characterized by R2.

FIG. 21 shows the kinetic parameters FcRn binding. Dissociation constants (KD), on-(kon) and off-rates (koff) were measured by BLI using recombinantly produced FcRn. Binding affinity to FcRn was determined at pH 6.0. Errors are standard errors from fitting using ForteBio data analysis software 9.1. Fitting quality is characterized by R2.

FIG. 22 shows the receptor binding BLI sensorgrams of unconjugated Fcabs, Cetuximab variants and respective MMAE conjugates (A) EGFR binding analysis. Association and dissociation were recorded at pH 7.4 and fitted by a 1:1 global full-fit binding model. (B) FcRn binding analysis. Association and dissociation of analytes were recorded at pH 6.0 and fitted by a 1:1 global partial-dissociation model. Fittings are shown in red. For each sensorgram, the highest concentration of analyte during association and its dilution factor are given.

Even without further embodiments, it is assumed that a person skilled in the art will be able to use the above description in the broadest scope. The preferred embodiments should therefore merely be regarded as descriptive disclosure which is absolutely not limiting in any way.

All the references cited herein are incorporated by reference in the disclosure of the invention hereby.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

Unless indicated otherwise, percent data denote percent by weight. All temperatures are indicated in degrees Celsius. “Conventional work-up”: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate, filtered and evaporated, and the product is purified by chromatography on silica gel and/or by crystallisation.

Rf values on silica gel; mass spectrometry: EI (electron impact ionisation): M⁺, FAB (fast atom bombardment): (M+H)⁺, THF (tetrahydrofuran), NMP (N-methlpyrrolidone), DMSO (dimethyl sulfoxide), EA (ethyl acetate), MeOH (methanol), TLC (thin-layer chromatography)

### List of Abbreviations

AUC Area under the plasma drug concentration-time curve
Cₘₐₓ Maximum plasma concentration
CL Clearance
CV Coefficient of variation
CYP Cytochrome P450
DMSO Dimethyl sulfoxide
F Bioavailability
fₐ Fraction absorbed
iv Intravenous
LC-MS/MS Liquid chromatography tandem mass spectrometry
LLOQ Lower limit of quantification
NC Not calculated
ND Not determined
PEG Polyethylene glycol
Pgp Permeability glycoprotein
PK Pharmacokinetic(s)
po Per os (oral)
t_{1/2} Half-life
tₘₐₓ Time at which maximum plasma concentration of drug is reached
UPLC Ultra performance liquid chromatography
Vₛₛ Volume of distribution (at steady state)
v/v Volume to volume

### EXAMPLES

### Example 1: Preparation of Fcabs, Controls and pHAb-Dye Labeled Constructs

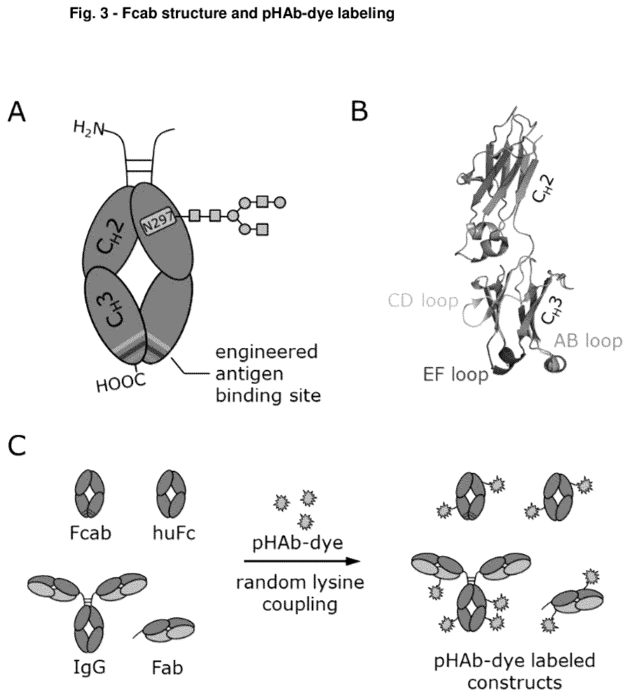

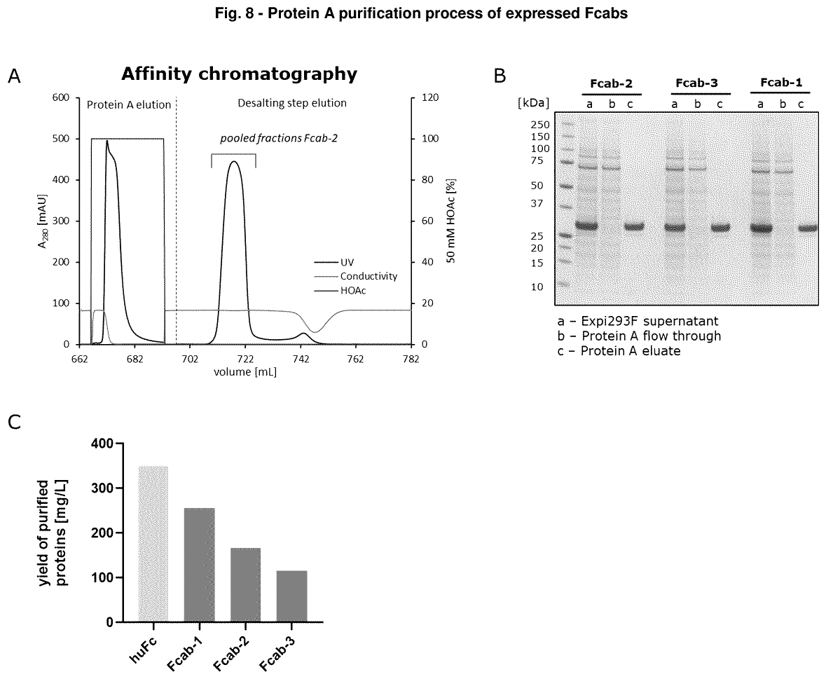

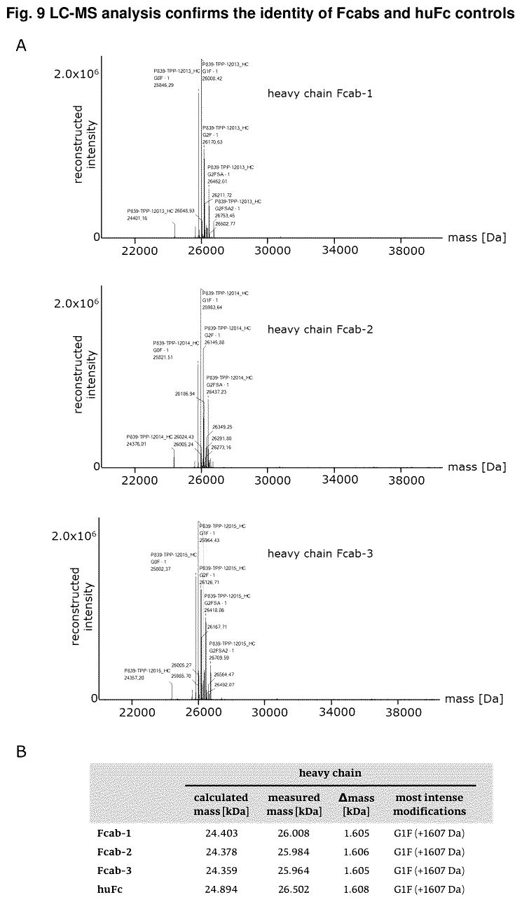

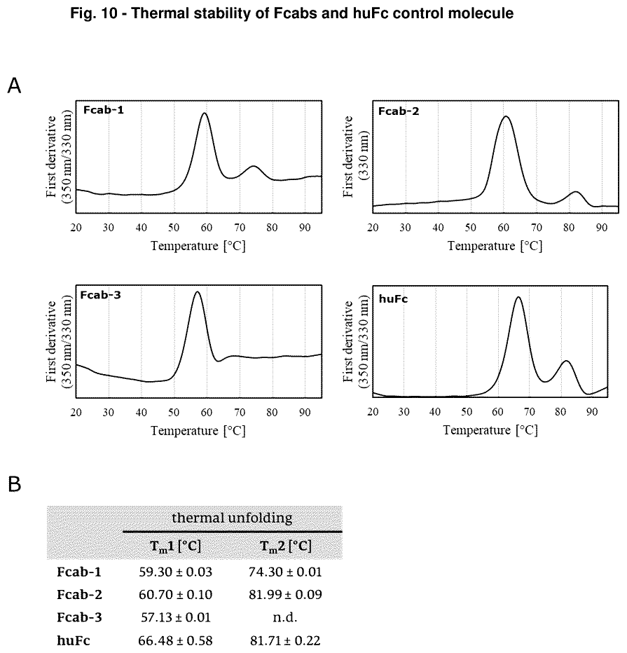

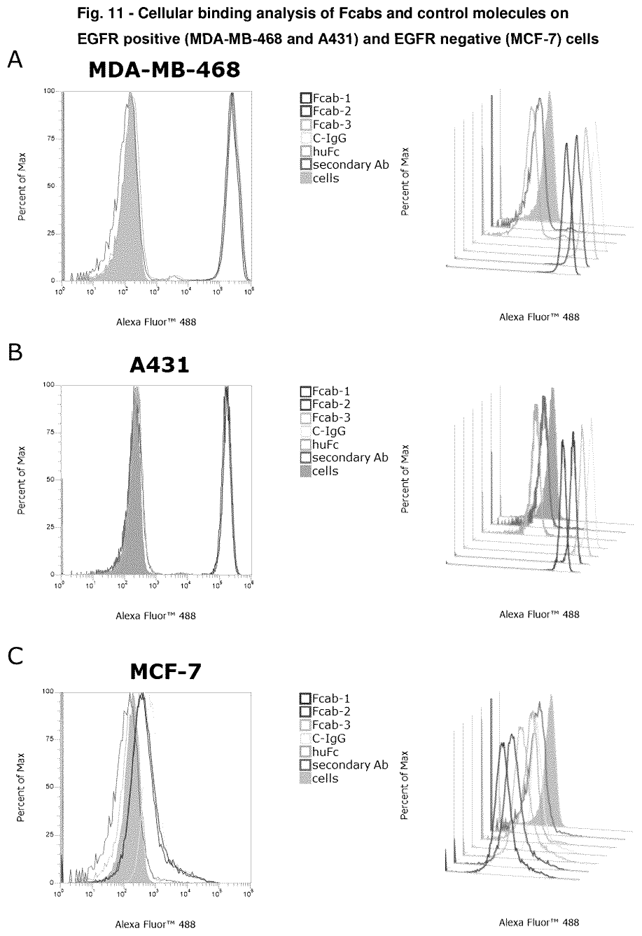

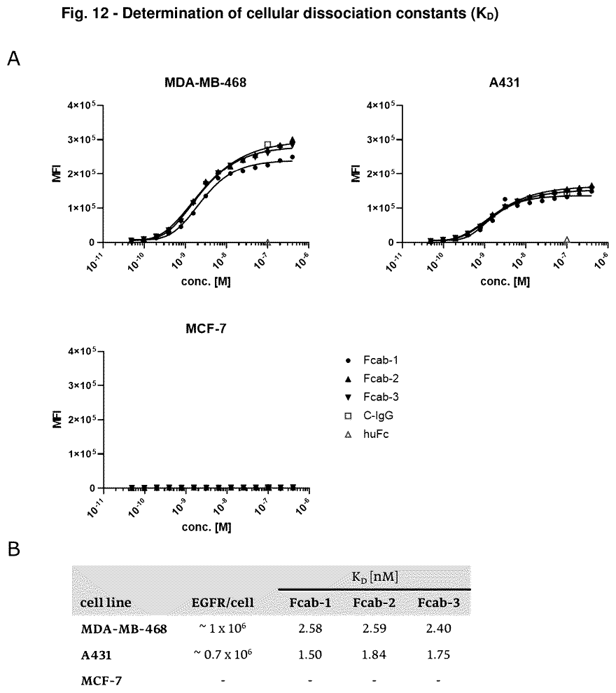

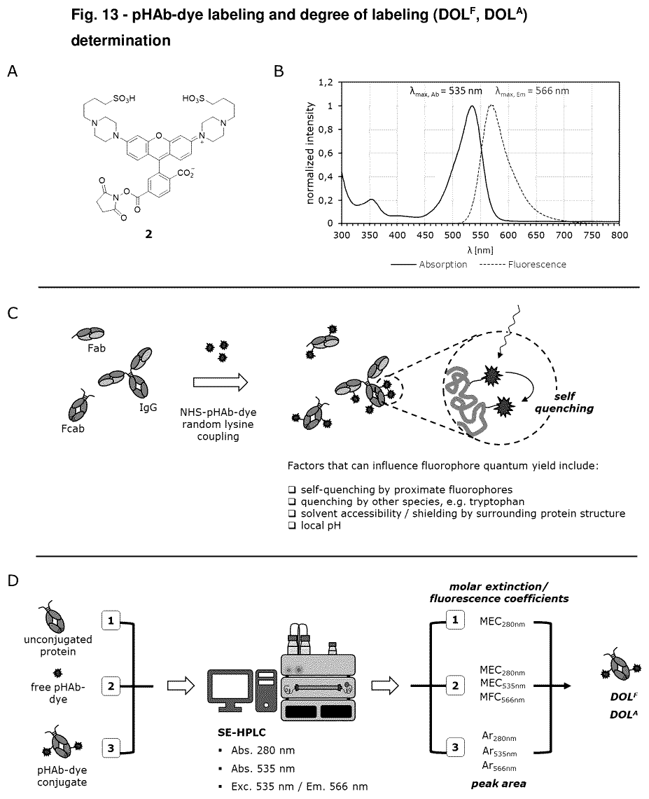

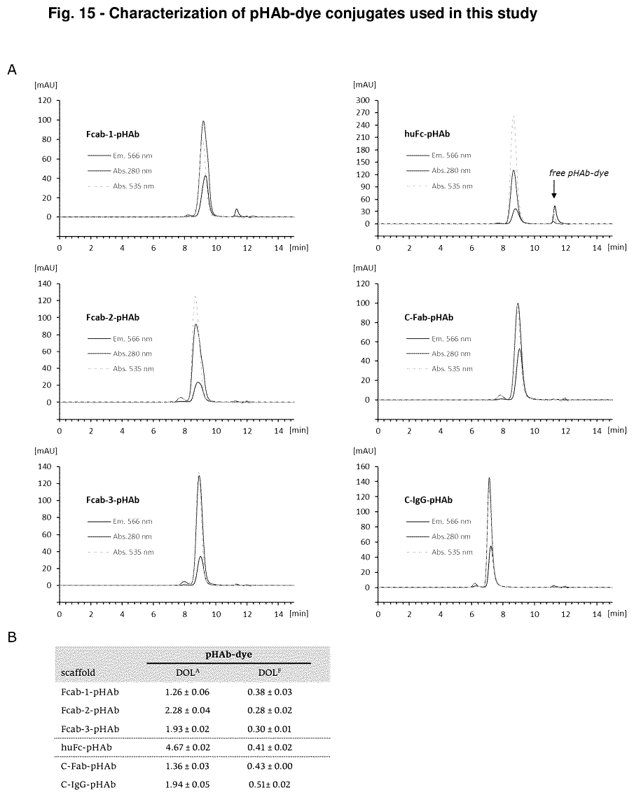

As a starting point, we selected three different EGFR-binding Fcabs (Fcab-1, Fcab-2, Fcab-3) from literature. For all three, single-digit nanomolar binding affinities to EGFR have been described (KD 0.7-2.6 nM). As a negative control, we included an unmodified human Fc (huFc) fragment. As EGFR-binding references, a Cetuximab-based full length IgG (C-IgG) and a Cetuximab-derived Fab (C-Fab) fragment, both equipped with a sortase A (SrtA) recognition motif (LPETG) at the C-terminal end of the light chain, were included. Fcabs and huFc were expressed with a D265A mutation^{[21,22]} to avoid Fcγ receptor (I, II, III) mediated cytotoxicity^{[23]} and purified by affinity chromatography (FIG. 8). Mass spectrometry analysis (LC-MS, FIG. 9) confirmed the identity of all molecules. Differential scanning fluorimetry showed lower unfolding transition midpoints (Tm) for Fcabs (e.g. T_{m,1} 59° C. vs. 66° C.; FIG. 10) indicating diminished but still acceptable thermal stability of Fcabs compared to huFc. Moreover, we evaluated functional and selective cellular binding of the purified constructs (FIG. 11, FIG. 12). For cellular uptake studies all constructs were labeled at random lysines via amine-coupling with a fluorescent dye (pHAb-dye) (FIG. 3C) that exhibits very low fluorescence at the neutral pH outside cells but strongly increased fluorescence at the acidic pH in endosomal and lysosomal compartments (FIG. 13). Successful pHAb-dye labeling and the absence of aggregates were confirmed by analytical size exclusion chromatography (SE-HPLC) (FIG. 15). To ensure comparability between individual fluorescence intensities resulting from differently labeled constructs, the fluorescence signals were normalized. A comprehensive description of the underlying experimental procedures can be found in the SI.

### Example 2: Cellular Uptake Studies

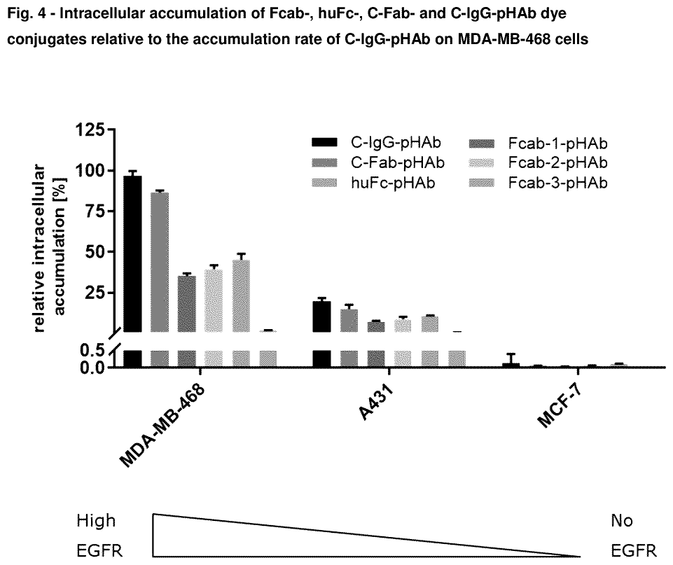

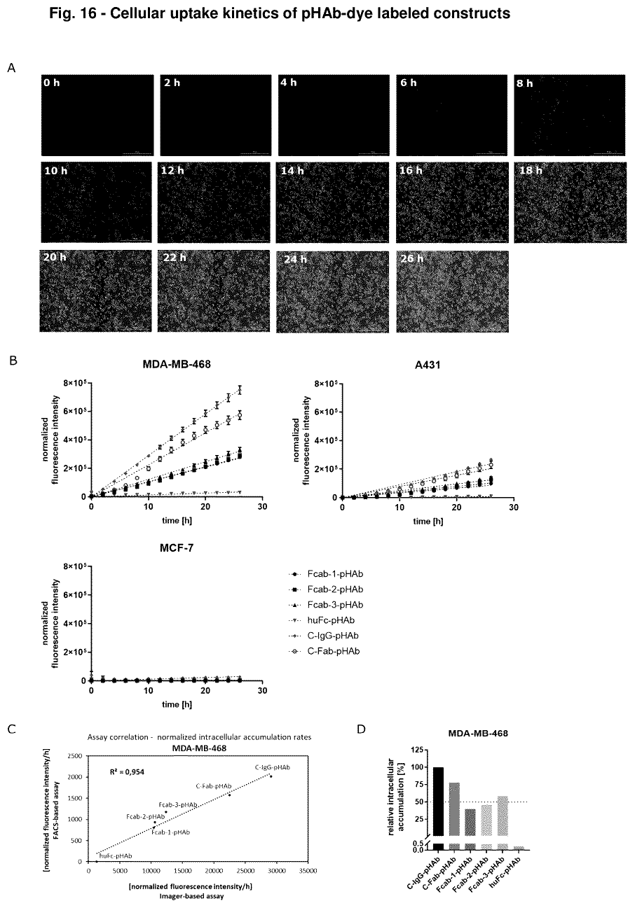

To assess the general suitability of EGFR-binding Fcabs for intracellular drug delivery, their uptake and accumulation into cancer cells were studied. Therefore, pHAb-dye labeled constructs were incubated on EGFR overexpressing (MDA-MB-468, A431) and EGFR negative (MCF-7) adherent cells and fluorescence was continuously measured over 26 hours (FIG. 16A). Fluorescence intensities were normalized to account for individual cell numbers and fluorescence intensities of labelled constructs. These intensities were plotted against the time to determine intracellular accumulation rates by linear regression (FIG. 16B). All accumulation rates were expressed relative to the rate of C-IgG-pHAb (C-IgG-pHAb on MDA-MB-468) which showed the fastest uptake (FIG. 4). Differences in accumulation between MDA-MB-468 and A431 cells directly correlated with the differences in specific EGFR expression density. Remarkably, all Fcabs underwent selective, EGFR-mediated cellular uptake. Interestingly, bivalent and monovalent controls C-IgG-pHAb and C-Fab-pHAb exhibited most pronounced internalization, which may arise from different epitopes addressed by Cetuximab than by the Fcabs. To measure the therapeutic threshold (IC₅₀) for respective Fcab-drug conjugates, Fcabs were conjugated to cytotoxic payloads and tested for their cell killing activity.

### Example 3: Generation of Fcab-Drug Conjugates

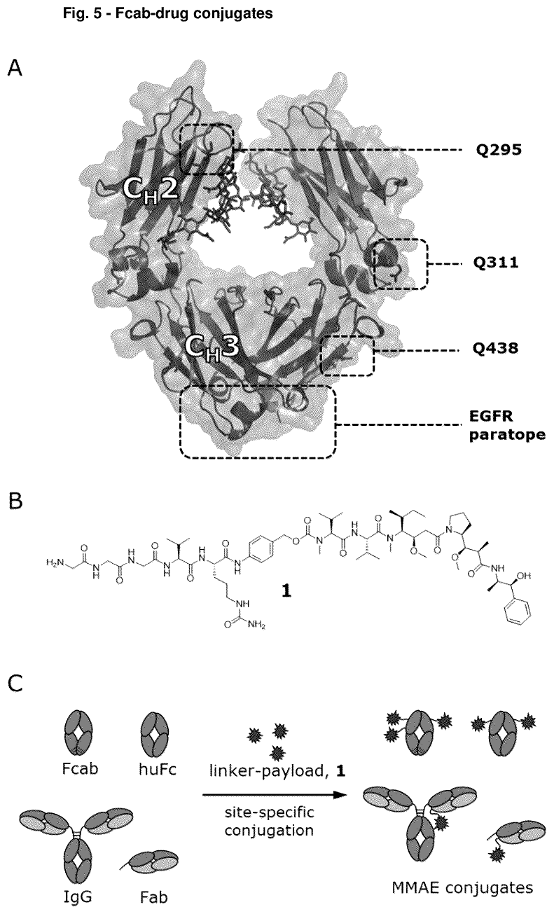

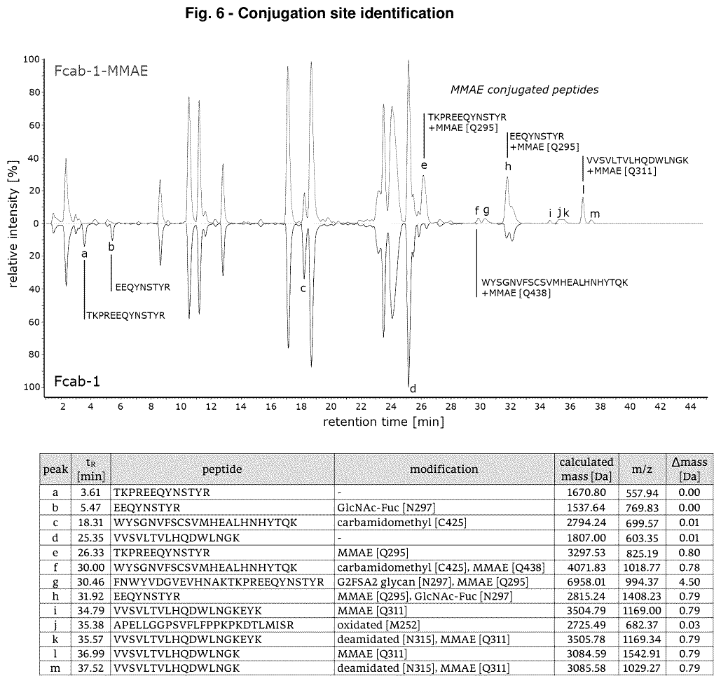

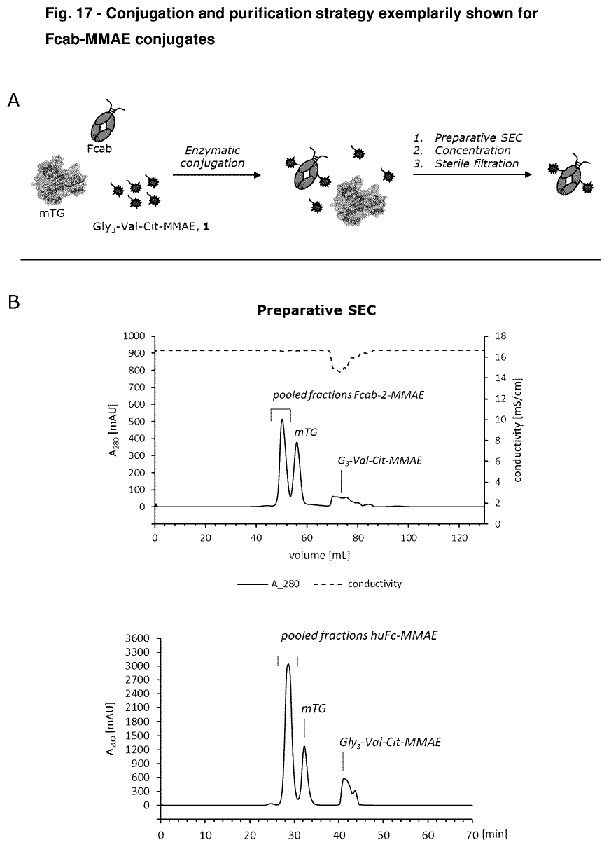

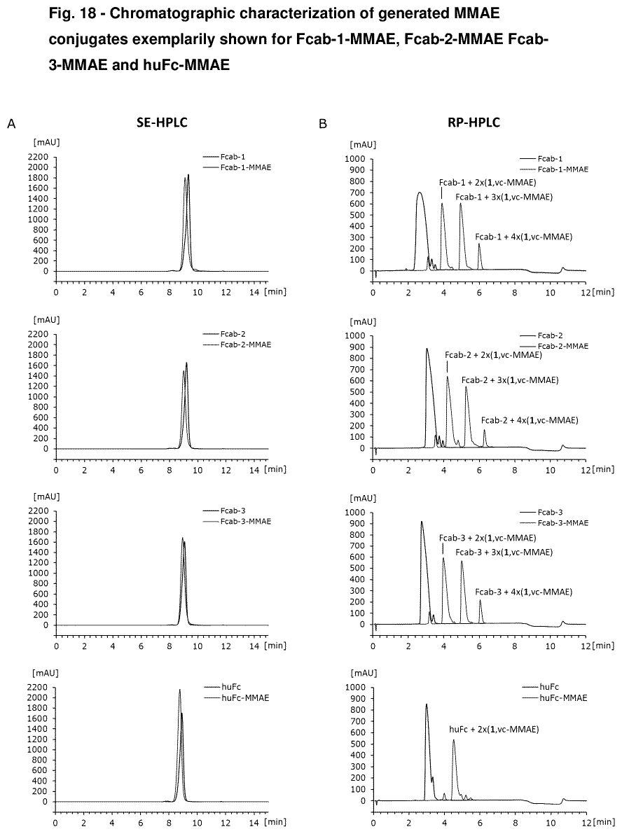

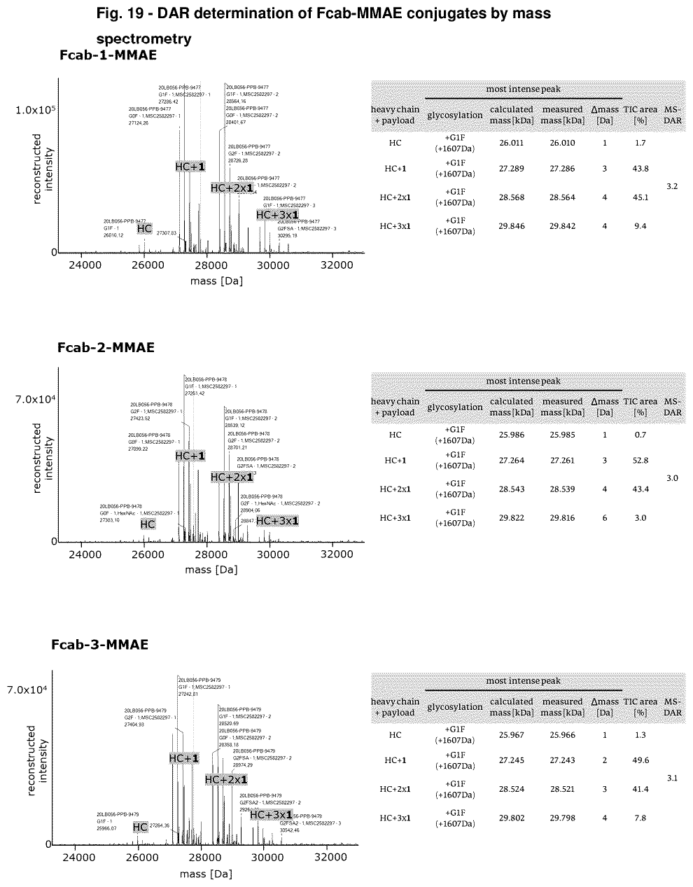

Fcab-1, Fcab-2, Fcab-3 and huFc were conjugated by an engineered transglutaminase (mTG)^{[27]} targeting Q295 with Val-Cit-PAB-MMAE possessing a triple glycine handle (1) (FIG. 5) (Table 1) and purified by preparative size exclusion chromatography (FIG. 17). C-Fab and C-IgG were conjugated to 1 by SrtA^{[28,29]} reaching DARs ranging DAR 0.8-1.1. Analytical SE-HPLC of purified conjugates showed the absence of high molecular weight species (FIG. 18A) (Table 1). Surprisingly, analyses via reversed phase chromatography (RP-HPLC) (FIG. 18B), hydrophobic interaction chromatography (HI-HPLC) (Table 4) and LC-MS (FIG. 19) revealed that all three Fcabs showed an elevated DAR between 2.7-2.9. This was not observed for huFc (DAR 2.0) indicating that substrate 1 had been coupled to additional residues in the Fcab scaffold only (Table 1). The additional conjugation sites could subsequently be identified via LC-MS peptide mapping as Q311 and Q438 in the constant C_{H}2 and C_{H}3 region (FIG. 5A) (FIG. 6). The disappearance of peaks assigned to unconjugated peptides containing Q295 in the digested Fcab-1-MMAE mixture indicated that Fcabs were nearly completely conjugated at position Q295 while only partially conjugated at position Q311 and Q438. As previous mTG conjugation to the same constant region in HER2 Fcabs or native IgGs did not lead to Q311 or Q438 conjugation[14], accessibility for mTG has most likely been driven by structural changes in adjacent regions. The changes can either be induced by the EGFR paratope inserted to the C_{H}3 region or by the missing hinge region of the EGFR Fcabs. Both, Q311 and Q438 lie within the solvent exposed exterior of the Fcab. Q311 is closely located to the FcRn and Q438 is closely located to the EGFR binding site. Consequently, conjugation at Q311 and Q438 could theoretically affect accessibility for serum proteases and interfere with FcRn and EGFR binding. To assess this notion, Fcab-1-MMAE, Fcab-2-MMAE and Fcab-3-MMAE were subsequently tested for FcRn and EGFR binding and serum stability.

TABLE 1
Overview of conjugated Fcabs and controls. Size refers to unconjugated
protein including the most abundant glycosylation pattern as
measured by LC-MS. DAR is given as a mean from RP-HPLC and
LC-MS analysis. SE-HPLC purity refers to the final drug conjugate
and was analyzed after a freeze-thaw cycle.

| | | conjugation | | | |
|---|---|---|---|---|---|
| | size | | | | SE-HPLC |
| conjugate | [kDa] | site | technique | DAR | purity [%] |
| Fcab-1- | 52.02 | Q295, Q311, | mTG | 2.9 | 96.7 |
| MMAE | | Q438 | | | |
| Fcab-2- | 51.07 | Q295, Q311, | mTG | 2.7 | 98.5 |
| MMAE | | Q438 | | | |
| Fcab-3- | 51.93 | Q295, Q311, | mTG | 2.8 | 99.7 |
| MMAE | | Q438 | | | |
| huFc-MMAE | 53.00 | Q295 | mTG | 2.0 | 100.0 |
| C-Fab-MMAE | 50.49 | LC-C-LPETG | SrtA | 0.8 | 99.5 |
| C-IgG-MMAE | 155.37 | LC-C-LPETG | SrtA | 1.1 | 99.5 |

### Example 4: Receptor Binding Properties of Fcab-Drug Conjugates

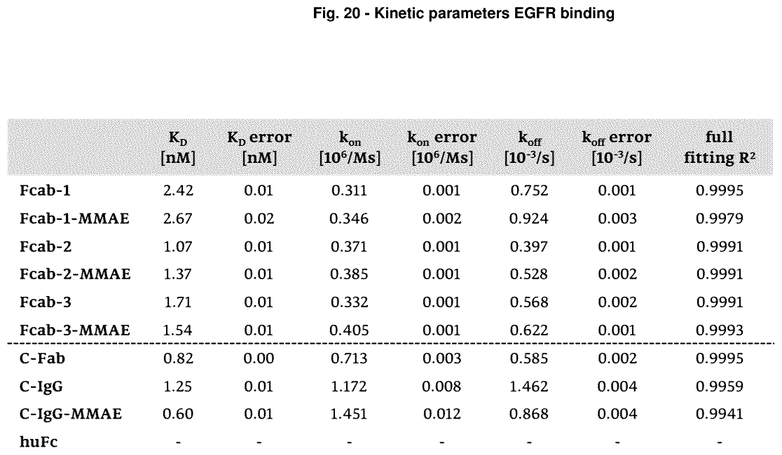

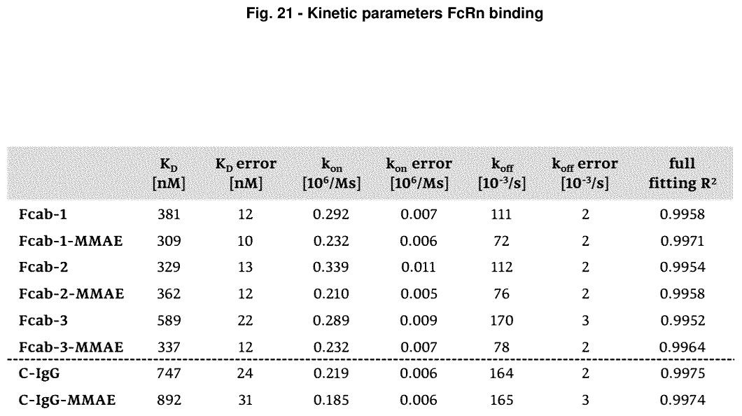

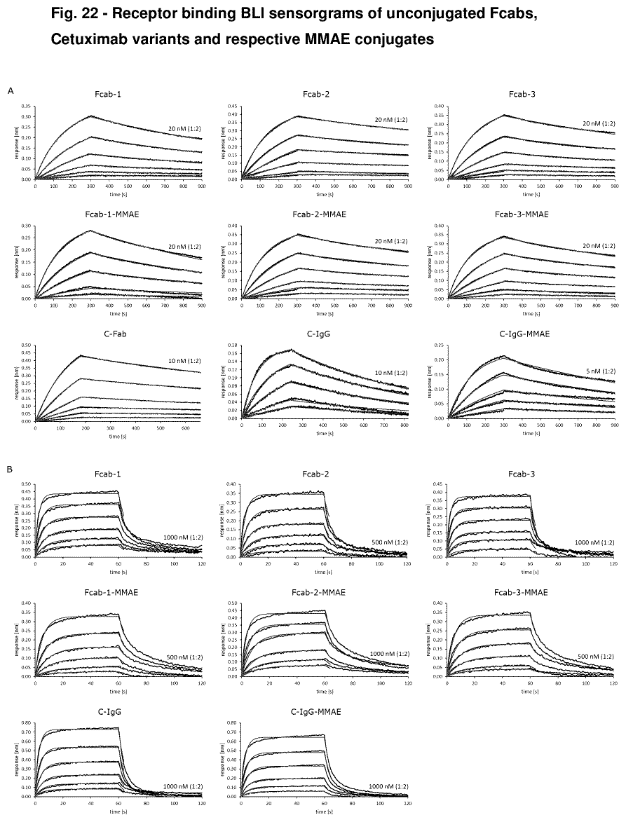

Fcab-MMAE conjugates were analyzed along with controls and non-conjugated parent molecules for their binding affinity to the target receptor EGFR and half-life extending FcRn (Table 2) (FIGS. 20-22). Biolayer interferometry (BLI) measurements revealed that EGFR and FcRn dissociation constants (KD) were not impaired by conjugation suggesting that attached payloads at positions Q438, Q311 and Q295 do not impact binding functionalities of both receptors.

TABLE 2
Binding affinity and serum stability of Fcab-drug conjugates and controls.
Dissociation constants (K_{D}) were measured by BLI. Binding to recombinantly
produced EGFR and FcRn were measured at pH 7.4 and pH 6.0, respectively.
Errors are standard errors from fitting using FortéBio data
analysis software 9.1. Free MMAE was measured via LC MS/MS after incubation
in mouse and human sera at 37° C. for 96 h (n = 3). Numbers
show the released fraction relative to initially conjugated MMAE.

| | non-conjugated | | MMAE-conjugates | | | |
|---|---|---|---|---|---|---|
| | K_{D} | K_{D} | K_{D} | K_{D} | free MMAE (% | |
| | (EGFR) | (FcRn) | (EGFR) | (FcRn) | total MMAE) | |
| conjugate | [nM] | [nM] | [nM] | [nM] | mouse | human |
| Fcab-1-MMAE | 2.42 ± 0.01 | 381 ± 12 | 2.67 ± 0.02 | 309 ± 10 | 1.3 | 0.3 |
| Fcab-2-MMAE | 1.07 ± 0.01 | 329 ± 13 | 1.37 ± 0.01 | 362 ± 12 | 1.1 | 0.0 |
| Fcab-3-MMAE | 1.71 ± 0.01 | 589 ± 22 | 1.54 ± 0.01 | 337 ± 12 | 0.9 | 0.0 |
| huFc-MMAE | 0.0 | n.d. | 0.0 | n.d. | n.d. | n.d. |
| C-Fab-MMAE | 0.82 ± 0.01 | — | n.d. | — | n.d. | n.d. |
| C-IgG-MMAE | 1.25 ± 0.01 | 747 ± 24 | 0.60 ± 0.01 | 892 ± 31 | n.d. | n.d. |

n.d.—not determined

### Example 5: Serum Stability of Fcab-Drug-Conjugates

Several studies have shown, that conjugate pharmacokinetics can strongly be influenced by premature cleavage of the linker that connects the protein with the cytotoxic drug.^{[30-32]} The Val-Cit linker motif is especially prone to cleavage by a carboxylesterase (mCes1c) that is present in mouse serum but absent in human serum.^{[33]} The extend of this instability heavily dependents on the chosen conjugation sites. When Fcab-MMAE conjugates were incubated in mouse and human serum for 96 h, no free MMAE could be detected for all constructs. This indicates that the Val-Cit linker motif is not accessible for mCes1c neither at position Q295 nor at the novel positions Q311 and Q438, hence all positions protect the conjugate from being cleaved prematurely (Table 2).

### Example 6: In Vitro Cytotoxicity of Fcab-Drug Conjugates

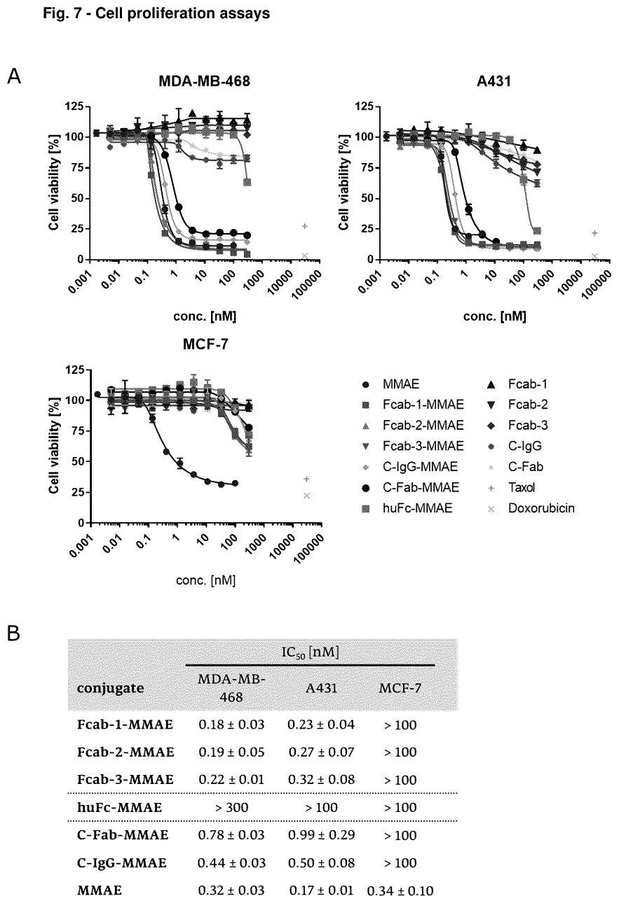

Next, we evaluated selective cell killing capabilities of the Fcab-drug conjugates in an in vitro cell proliferation assay (FIG. 7). All Fcab-drug conjugates showed similar sub-nanomolar inhibitory activity on EGFR positive MDA-MB-468 and A431 cells (IC₅₀ 0.18-0.22 nM and 0.23-0.32 nM, respectively) while toxicity against EGFR negative MCF-7 cells was decreased by several orders of magnitude (IC₅₀>100 nM) indicating strong target-dependent cell killing. Non-targeting huFc-MMAE showed low toxicity as well (MDA-MB-468: IC₅₀>300 nM; A431 and MCF-7: IC₅₀>100 nM) confirming that Fcab-MMAE toxicity is primarily driven by specific receptor-mediated uptake. In line with higher cellular uptake on MDA-MB-468 cells compared to A431 cells (FIG. 5), Fcab-drug conjugates and Cetuximab controls showed higher activity on MDA-MB-468 cells compared to A431 (FIG. 7B). Furthermore, higher potency of Fcab-drug conjugates on EGFR positive cells (IC₅₀ 0.18-0.32 nM) compared to C-Fab-MMAE (IC₅₀ 0.78-0.99 nM) or C-IgG-MMAE (IC₅₀ 0.44-0.50 nM) suggested that reduced intracellular accumulation of Fcabs (FIG. 5) may be compensated by their higher DAR enabled by additional conjugation to Q311 and Q438 (2.7-2.9 versus 0.8 and 1.1, respectively). Overall, the results indicate that in vitro potency of Fcab-drug conjugates is in the typical sub-nanomolar range of ADCs despite their monovalent binding mode.

### Example 7: Cellular Uptake Assay

For our cellular uptake study we used a pH-dependent fluorophore (pHAb-dye) based assay.^{[35]} We directly labeled constructs with pHAb-dye by applying random lysine coupling and acquired their intracellular accumulation kinetics on cells by measuring the fluorescence increase over time generated when constructs reach the acidic endosome and/or lysosome. Importantly, the fluorescence of randomly coupled pHAb-dye molecules could be altered by pHAb-dye local molecular environment (FIG. 13C) probably resulting in a non-linear relationship between the number of coupled pHAb-dye molecules and the individual fluorescence of a construct.^{[35-37]} To consider this, we developed a method similar to Wang et al. to derive a fluorescence- and absorption-based degree of pHAb-dye labeling (DOL^{F} and DOL^{A}) from SE-HPLC data.^{[38]} The SE-HPLC method has the advantage to simultaneously analyze DOLF and DOLA as well as aggregation and purification (free pHAb-dye) status of the labeled construct. The DOLF value of each pHAb-dye labeled construct can then be used to normalize fluorescence values of intracellular accumulation kinetics. In the following a detailed description of the method is given. FIG. 13D summarizes the method in short.

### SE-HPLC Analysis

SE-HPLC was performed on a 1260 Infinity device from Agilent Technologies equipped with a diode array (DAD) and a fluorescence (FLD) detector module and either a TSKgel SuperSW3000 or a SuperSW2000 column. The mobile phase consisted of 50 mM sodium phosphate, 400 mM sodium perchlorate, pH 6.3 and its flow rate was set to 0.35 mL/min. The DAD was set to detect absorption at 280 nm (aromatic amino acids) and 535 nm (pHAb-dye). The excitation and emission wavelengths of the FLD were set to 535 nm and 566 nm to record fluorescence of pHAb-dye (FIG. 13B).

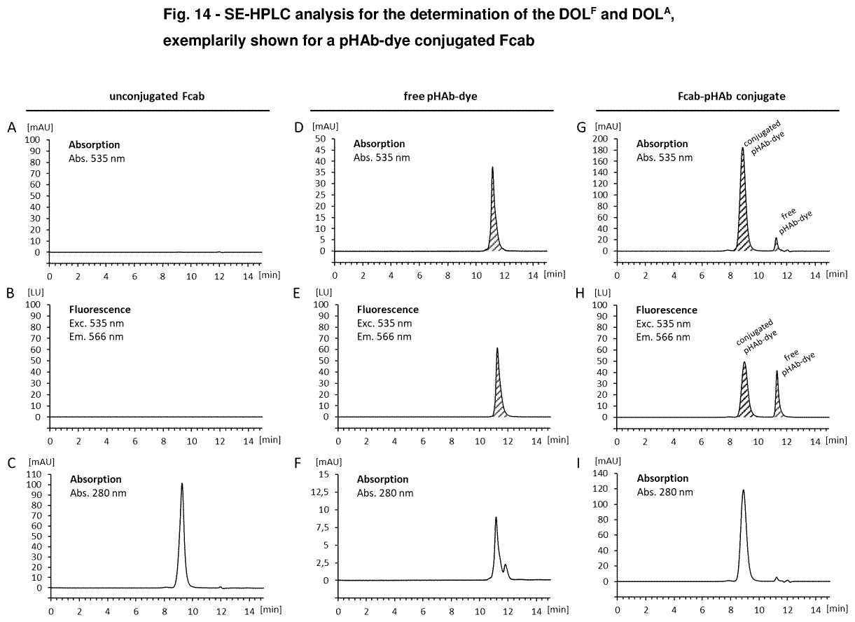

Free pHAb-dye, pHAb-dye conjugated proteins and the corresponding unconjugated proteins were then analyzed by SE-HPLC. FIG. 14 depicts exemplarily the resulting chromatograms for unconjugated protein, free pHAb-dye and pHAb-dye conjugated protein. In a next step, the molar extinction or fluorescence coefficients were derived from these data.

Calculating Molar Extinction/Fluorescence Coefficient (MEC/MFC) of Unconjugated Protein and Free pHAb-Dye from SE-HPLC Peak Area

MEC/MFC of unconjugated protein and free pHAb-dye were calculated from SE-HPLC peak areas. Exemplary chromatograms can be found in Error! Reference source not found. C (unconjugated protein) and FIG. 14D-F (free pHAb-dye). Relevant peaks were integrated using the ChemStation analysis software by Agilent. From the corresponding peak area, the MEC₂₈₀ₙₘ of the unconjugated protein and the MEC₅₃₅ₙₘ/MFC₅₆₆ₙₘ of free pHAb-dye could be determined applying the following equation derived from Wang et al.^{[5]}

$${ε}_{i} = \frac{Ar \cdot F}{l \cdot c \cdot {v}_{inj}} \qquad ( 1 )$$

where εᵢ is the MEC or MFC at wavelength λᵢ, Ar is the calculated peak area, F is the SE-HPLC flow rate, l is the flow cell path length, c is the concentration of the injected sample, and uᵢₙⱼ is the injected sample volume. Table 3 summarizes the resulting MEC and MFC of unconjugated proteins and free pHAb-dye.

TABLE 3
Molar extinction/fluorescence coefficient at different wavelengths.
MEC and MFC are given as the mean ± SD. Different volumes of
unconjugated protein or free pHAb-dye solution were injected in
three consecutive SE-HPLC runs and the resulting peak areas were
used to calculate MEC or MFC from equation (1). For example, when
injecting Vᵢₙⱼ = 7.5 μL of c = 18.3 μM Fcab a single
peak with a peak area Ar_{280 nm} of 1825 eluted at 9.3 min (FIG. 14C).
With a constant SE-HPLC flow rate of F = 5.8 μL/s, a MEC_{280 nm}
of 77122 (mM · cm)⁻¹ was calculated.

| | MEC_{280 nm} | MEC_{535 nm} | MFC_{566 nm} |
|---|---|---|---|
| analyte | [mM · cm]⁻¹ | [mM · cm]⁻¹ | [mM · cm]⁻¹ |
| Fcab-1 | 94582 ± 685 | — | — |
| Fcab-2 | 94608 ± 1 | — | — |
| Fcab-3 | 96964 ± 155 | — | — |
| huFc | 75217 ± 306 | — | — |
| C-Fab | 72828 ± 26 | — | — |
| C-IgG | 107610 ± 182 | — | — |
| free pHAb-dye | 14825 ± 257 | 60813 ± 823 | 103746 ± 3003 |

Calculating the DOL^{F} and DOL^{A} from SE-HPLC Peak Area of pHAb-Dye Conjugated Protein and MEC/MFC

To calculate the DOLF value from a pHAb-dye labeled construct, the molar amount of conjugated fluorescent pHAb-dye (N_{pHAb,566nm}) and protein (n_{protein,280nm}) was calculated first. Therefore, pHAb-dye labeled constructs were analyzed by SE-HPLC and the absorption and fluorescence peak area of conjugated pHAb-dye (FIG. 14G-H peaks marked in red), as well as peak area from absorption at 280 nm (FIG. 141) were calculated. Absorption at 280 nm is caused not only by the protein structure but also by the conjugated pHAb-dye (FIG. 14F). To calculate the amount of injected protein from this peak, the peak area that is contributed by pHAb-dye must be subtracted. The portion of pHAb-dye absorption at 280 nm can be derived from its peak area at 535 nm (Ar_{535 nm}) and subtracted from total peak area at 280 nm (Ar₂₈₀ₙₘ) as shown in equation (2)

$${Ar}_{280 nm , corrected} = {Ar}_{280 nm} - ( {Ar}_{535 nm} \cdot \frac{{MEC}_{pHAb , 280 nm}}{{MEC}_{pHAb , 535 nm}} ) \qquad ( 2 )$$

Subsequently, the amount of injected protein can be calculated from the corrected peak area (Ar_{280nm,corrected}) by equation (3)

$${n}_{protein , 280 nm} = \frac{{Ar}_{280 nm , corrected} \cdot F}{{MEC}_{protein , 280 nm}} \qquad ( 3 )$$

Similarly, the amount of conjugated fluorescent pHAb-dye can be calculated from the peak area of its fluorescence signal at 566 nm (Ar₅₆₆ₙₘ):

$${n}_{pHAb , 566 nm} = \frac{{Ar}_{566 nm} \cdot F}{{MFC}_{pHAb , 566 nm}} \qquad ( 4 )$$

The ratio between the amount of conjugated fluorescent pHAb-dye molecules and protein defines the DOL^{F} value for the individual fluorescence of a construct:

$${DOL}^{F} = \frac{{n}_{pHAb , 566 nm}}{{n}_{protein , 280 nm}} \qquad ( 5 )$$

In analogy, the absolute amount of conjugated pHAb-dye molecules per protein (DOL^{A}) can be calculated from the peak area of pHAb-dye absorption at 535 nm applying equation (6) and (7).

$${n}_{pHAb , 535 nm} = \frac{{Ar}_{535 nm} \cdot F}{{MEC}_{pHAb , 535 nm}} \qquad ( 6 )$$

$${DOL}^{A} = \frac{{n}_{pHAb , 535 nm}}{{n}_{protein , 280 nm}} \qquad ( 7 )$$

The SE-HPLC chromatograms of relevant pHAb-dye conjugates as well as DOL^{F} and DOL^{A} values are shown in Error! Reference source not found. In line with our expectations, the number of fluorescent pHAb-dye molecules per protein (DOL^{F}) is lower compared to the absolute number of conjugated pHAb-dye molecules (DOL^{A}). Since fluorescence represents the readout of the cellular uptake assay, fluorescence values can be normalized to their DOL^{F} value to account for the individual fluorescence of pHAb-dye labeled constructs. Consequently, the DOL^{F} and cell number normalized intracellular accumulation rates allow for comparability.

### Example 8: Material and Methods

### Preparation of Antibody Fragments:

Amino acid sequences of Fcabs were taken from literature. Sequences of modified Fcabs (D265A) are given along with modified huFc (D265A) and Cetuximab sequences (SrtA tag) in the SI. Encoding sequences were ordered as codon-optimized versions and cloned into pTT5 vector for mammalian expression (GeneArt, Thermo Fisher Scientific). Fcabs and huFc controls were expressed by transient transfection of Expi293F™ cells following the manufacturer's instructions and the supernatants were harvested after 5 days post transfection. C-Fab contained a His6-Tag for purification and was dialyzed against phosphate-buffered saline (PBS) pH 7.4 before purification by immobilized metal affinity chromatography (1 mL HisTrap™ HP, GE Healthcare) using an ÄKTA Pure device (GE Healthcare). Fcabs, huFc and C IgG were purified by protein A affinity chromatography using HiTrap™ Mab Select SuRe 5 mL columns (GE Healthcare) and subsequently formulated in PBS pH 6.8 using HiPrep™ 26/10 desalting columns. Antibody purity was analyzed by analytical SE-HPLC using a TSKgel® SuperSW3000 column (Tosoh Bioscience) and by SDS gel electrophoresis. Identity of proteins was confirmed via intact mass analysis by LC-MS using an Exion LC and Triple TOF® 6600+ mass spectrometer (AB Sciex). Proteins were concentrated using Ultra centrifugal filter units (3K MWCO, Amicon®), sterile filtered and protein concentration was determined by UV-VIS spectroscopy at 280 nm. Proteins were snap-frozen in liquid nitrogen and stored at 80° C.

### Preparation of MMAE Conjugates:

Fcabs and huFc were conjugated to drug-linker Gly3-Val-Cit-PAB-MMAE (1, Levena) using a genetically engineered mTG. mTG-mediated antibody conjugation was performed using 5 mg/mL Fcabs/huFc, 20 molar equivalents of drug-linker and 60 U/mL mTG in PBS pH 6.8 with up to 10% DMSO. Reaction mixes were incubated at 37 ºC for 18 h with gentle shaking, chilled to 10° C. and purified by preparative size exclusion chromatography (SEC).

For SrtA conjugation, C IgG or C Fab (5 mg/mL) were formulated in 150 mM NaCl, 50 mM Tris-HCl, 5 mM CaCl2 pH 7.5. SrtA^{[29]} was added to a final concentration of 13 μM along with 10 equivalents of Gly3 Val-Cit-PAB-MMAE (1) per SrtA recognition motif. The reaction mixture was incubated for 90 min at 25° C., stopped by the addition of EDTA (final 10 mM) and purified by preparative SEC. Preparative SEC was performed using either a Superdex™ 200 Increase 10/300 GL, Superdex™ 75 10/30 GL or a Superdex™ 200 prep grade 16/60 column in a 1260 liquid chromatography system (Agilent Technologies) or an ÄKTA Avant device (GE Healthcare) with PBS pH 6.8 as running buffer. Purified conjugates were concentrated using Ultra centrifugal filter units (10K MWCO, Amicon®), sterile filtered and protein concentration was determined by UV-VIS spectroscopy at 280 nm. The purified conjugates were subjected to analysis by SE-HPLC and DAR determination (RP HPLC, LC-MS) as described elsewhere[27], snap-frozen in liquid nitrogen and stored at −80° C.

Preparation of pHAb-dye Conjugates:

Fcabs, huFc and Cetuximab controls were formulated in 10 mM sodium-bicarbonate buffer pH 8.5. pHAb amine reactive dye (10 mg/mL 1:1 (v/v) DMSO/H2O, Promega) was either added at a 2:1 molar ratio (pHAb:antibody) (Fcab 1, Fcab 2, Fcab 3, C IgG, C Fab) or a 10:1 molar ratio (huFc), followed by incubation at 25° C., 450 rpm for 1 h in the absence of light. Excess dye was removed by dulbecco's phosphate buffered saline (DPBS) equilibrated Zeba™ Spin desalting columns (ThermoFisher Scientific) according to the manufacturer's instructions. Aggregation of pHAb-dye conjugates and fluorescence degree of labeling (DOLF) were determined by an SE-HPLC method described in the SI. Peptide Mapping: Fcab-1 and Fcab-1-MMAE were deglycosylated with GlycINATOR (Genovis) according to the instruction manual. Deglycosylated molecules were then reduced with 10 mM DTT for 30 min at 56° C. and alkylated with 55 mM iodoacetamide for 30 min at room temperature in the dark. 10 μg protein was digested with 0.5 μg trypsin (mass spectrometry grade, Promega) at 37° ° C. overnight.

LC-MS analysis was performed using an Exion HPLC system coupled to a TripleTOF 6600+ mass spectrometer (Sciex). 7.5 μg peptide solution was loaded onto an Aeris PEPTIDE XB-C18 column (Phenomenex, part no. 00F-4506-AN) and eluted with a linear gradient from 5% to 50% buffer B (acetonitrile, 0.1% formic acid; buffer A: water, 0.1% formic acid) within 49 min. Data were acquired with positive polarity and in a TOF-MS mass range from 350 to 2500 m/z and a TOF-MS/MS mass range from 50 to 2500 m/z. Other instrument settings were as follows: ion spray voltage 5.5 kV, source temperature 450° C., accumulation time 0.25 s for TOF-MS and 0.08 s for TOF-MS/MS, gas1 45 psi, gas2 45 psi, curtain gas 35 psi, declustering potential 80 V, and collision energy was set to dynamic. Data were processed with Genedata Expressionist.

### Cell Culture:

Human cancer cell lines were obtained from the American Type Culture Collection (EGFR positive: MDA MB 468, A431; EGFR negative: MCF 7) and maintained according to standard culture conditions (37° C., 5% CO2, 95% humidity). A431 and MCF 7 cells were cultured in DMEM high glucose medium supplemented with 10% fetal bovine serum (FBS), 2 mM L-glutamine and 1 mM sodium pyruvate. MDA-MBA-468 cells were cultured in Roswell Park Memorial Institute (RPMI) 1640 medium supplemented with 10% FBS, 2 mM L-glutamine and 1 mM sodium pyruvate. For sub-culturing, adherent grown cells were detached by adding 0.05% trypsin-EDTA, diluted with fresh medium and transferred into a new culturing flask.

### Cellular Uptake:

Cells were centrifuged at 500×g for 5 min, the supernatant was discarded, and cells were resuspended in the respective medium without phenol red at 300,000 cells/mL. The cell suspension (40 μL/well) was seeded into a black 384 clear bottom plate followed by incubation (37° C., 5% CO2) in a humid chamber overnight. pHAb-dye labeled proteins were supplemented with 0.3% Tween-20 (final), diluted to 3 UM and added to the cells in triplicates (final 100 nM) using a D300e digital dispenser (Tecan). The cells were immediately transferred to a Cytation 5 cell imaging reader (BioTek) equipped with DAPI and RFP filter cubes and a BioSpa 8 automated incubator (BioTek). Brightfield (objective: 10×, LED intensity: 10, integration time: 13 msec, camera gain: 24) and RFP channel images (ex.: 531 nm, em.: 593 nm, LED intensity: 10, integration time: 50 msec, camera gain: 24) were taken every 2 h over a period of 26 h. About 30 min before the 26 h measurement, the plate was removed from the BioSpa 8 device and 1 μg/mL Hoechst 33342 (ThermoFisher Scientific) was added via a Tecan D300e digital dispenser for an additional 26 h endpoint DAPI image. Images were processed by the BioTek gen5 data analysis software. The sum of the integrated pHAb dye fluorescence intensities of each image was normalized to the number of cells determined in the DAPI channel and subtracted by the sum of the integrated RFP signal at 0 h (background signal). The cell number and background normalized intensities were divided by the pHAb-dye DOLF of each construct and plotted against the time. Normalized data was fitted by linear regression in GraphPad Prism (GraphPad Software, Inc.) and intracellular accumulation rates (slopes) were derived. Finally, the relative intracellular accumulation was calculated for each construct with respect to the highest intracellular accumulation rate (here, C IgG-pHAb on MDA MB 468 was set 100%).

### FcRn and EGFR Binding:

Kinetic parameters of Fcabs, Cetuximab variants and their respective MMAE conjugates were determined by BLI using the Octet® RED96 system (ForteBio, Pall) at 30° C. and 1,000 rpm agitation speed.

For EGFR binding analysis, Fcab variants (10 μg/mL in DPBS), C IgG (2.5 μg/mL in DPBS) and respective MMAE conjugates were loaded onto anti-human IgG Fc capture biosensors (AHC) for 60-180 s. C Fab (2.5 μg/mL in DPBS) was loaded onto anti human Fab C_{H}1 2nd Generation biosensors (FAB2G) for 180 s. Biosensors were then transferred into kinetics buffer (DPBS pH 7.4, 0.02% Tween 20 and 0.1% bovine serum albumin) and incubated for 60 s followed by an association step to EGFR-His6 (produced in-house). EGFR-His6 was serially diluted in kinetics buffer in a concentration range varying from 20 nM to 0.313 nM. Association was monitored for 180 s, 240 s or 300 s followed by a dissociation step in kinetics buffer for 600 s to determine kon and koff values. EGFR-His6 was replaced by kinetics buffer, serving as a negative control and reference. Respective non-binding huFc was used as negative control in each experiment. The buffer reference measurement (control curve) was subtracted from antibody measurements for data fitting and kinetics parameter were determined by using ForteBio data analysis software 12.0 applying a 1:1 global full-fit binding model after Savitzky-Golay filtering. The FcRn binding assay was performed as described elsewhere.

### Serum Stability:

The serum stability assay was conducted as previously described^{[27]} applying some minor modifications: MMAE conjugates were incubated at a final concentration of 5 μM conjugated MMAE (considering the DAR of each construct) in human and mouse serum. Moreover, serum samples were supplemented with 5 μM deuterated D8-MMAE internal standard prior to 96 h serum incubation.

### Cell Proliferation Assay:

For the evaluation of C IgG-, C Fab- and Fcab MMAE conjugates and related compounds, 40 μL of viable cell suspension were seeded into opaque 384-well plates (MDA MB 468: 2500 cells/well, A431: 9000 cells/well, MCF 7: 5000 cells/well) followed by incubation (37° C., 5% CO2) in a humid chamber overnight. Test compounds were added using a D300e digital dispenser (Tecan). Free MMAE, and protein/protein-conjugate solutions were supplemented with 0.3% Tween 20 (final) and diluted to 6 μM (MMAE) or 10 μM (proteins). All wells were normalized to the maximum amount of Tween 20 added. Cell viability was determined after 4 d using Cell Titer Glo reagent (Promega) according to the manufacturer's instructions. Luminescence values were normalized to luminescence of non-treated cells and dose-response was fitted using the asymmetric (five parameter) fitting function of GraphPad Prism (GraphPad Software, Inc.) to derive IC50 values.

### Example 9: Injection Vials

A solution of 100 g of a conjugate of the present invention and 5 g of disodium hydrogenphosphate in 3 l of bidistilled water is adjusted to pH 6.5 using 2 N hydrochloric acid, filtered under sterile conditions, transferred into injection vials, lyophilised under sterile conditions and sealed under sterile conditions. Each injection vial contains 5 mg of a conjugate of the present invention.

### Example 10: Solution

A solution is prepared from 1 g of a conjugate of the present invention, 9.38 g of NaH₂PO₄ 2 H₂O, 28.48 g of Na₂HPO₄·12 H₂O and 0.1 g of benzalkonium chloride in 940 ml of bidistilled water. The pH is adjusted to 6.8, and the solution is made up to 1 l and sterilised by irradiation.

### Example 11: Ampoules

A solution of 1 kg of a conjugate of the present invention in 60 I of bidistilled water is filtered under sterile conditions, transferred into ampoules, lyophilised under sterile conditions and sealed under sterile conditions. Each ampoule contains 10 mg of a conjugate of the present invention.

### Example 12: Amino Acid Sequences of Expressed Proteins

D265A, [symbol] , [symbol] , [symbol]
Fcab-1 (modified FS1-60^{[1]}):
SEQ ID No. 1
APELLGGPSVFLFPPKPKDTLMISRTPEVTCVVVAVSHEDPEVKF
NWYVDGVEVHNAKTKPREE [symbol] YNSTYRVVSVLTVLH [symbol] DWLNGKEYK
CKVSNKALPAPIEKTISKAKGQPREPQVYTLPPSRDELDEGGPVS
LTCLVKGFYPSDIAVEWESTYGPENNYKTTPPVLDSDGSFFLYSR
LTVSHWRWYSGNVFSCSVMHEALHNHYT [symbol] KSLSLSPG
Fcab-2 (modified FS1-65^{[1]}):
SEQ ID No. 2
APELLGGPSVFLFPPKPKDTLMISRTPEVTCVVVAVSHEDPEVKF
NWYVDGVEVHNAKTKPREE [symbol] YNSTYRVVSVLTVLH [symbol] DWLNGKEYK
CKVSNKALPAPIEKTISKAKGQPREPQVYTLPPSRDELDEGGPVS
LTCLVKGFYPSDIAVEWESTYGPENNYKTTPPVLDSDGSFFLYSK
LTVSYWRWVKGNVFSCSVMHEALHNHYT [symbol] KSLSLSPG
Fcab-3 (modified FS1-67^{[1]}):
SEQ ID No. 3
APELLGGPSVFLFPPKPKDTLMISRTPEVTCVVVAVSHEDPEVKF
NWYVDGVEVHNAKTKPREE [symbol] YNSTYRVVSVLTVLH [symbol] DWLNGKEYK
CKVSNKALPAPIEKTISKAKGQPREPQVYTLPPSRDETDDGPVSL
TCLVKGFYPSDIAVEWESTYGPENNYKTTPPVLDSDGSFFLYSKL
TVSYWRWYKGNVFSCSVMHEALHNHYT [symbol] KSLSLSPG
Fcab-4 (FS1-60^{[1]}):
SEQ ID No. 4
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDELDEGGPVS
LTCLVKGFYPSDIAVEWESTYGPENNYKTTPPVLDSDGSFFLYSR
LTVSHWRWYSGNVFSCSVMHEALHNHYTQKSLSLSPG
Fcab-5 (FS1-65^{[1]}):
SEQ ID No. 5
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDELDEGGPVS
LTCLVKGFYPSDIAVEWESTYGPENNYKTTPPVLDSDGSFFLYSK
LTVSYWRWVKGNVFSCSVMHEALHNHYTQKSLSLSPG
Fcab-6 (FS1-67^{[1]}):
SEQ ID No. 6
APELLGGPSVFLFPPKPKDTLMISRTPEVTCVVVDVSHEDPEVKF
NWYVDGVEVHNAKTKPREEQYNSTYRVVSVLTVLHQDWLNGKEYK
CKVSNKALPAPIEKTISKAKGQPREPQVYTLPPSRDETDDGPVSL
TCLVKGFYPSDIAVEWESTYGPENNYKTTPPVLDSDGSFFLYSKL
TVSYWRWYKGNVFSCSVMHEALHNHYTQKSLSLSPG
huFc (modified human IgG1 Fc
fragment):
SEQ ID No. 7
TCPPCPAPELLGGPSVFLFPPKPKDTLMISRTPEVTCVVVAVSHE
DPEVKFNWYVDGVEVHNAKTKPREE [symbol] YNSTYRVVSVLTVLHQDWL
NGKEYKCKVSNKALPAPIEKTISKAKGQPREPQVYTLPPSRDELT
KNQVSLTCLVKGFYPSDIAVEWESNGQPENNYKTTPPVLDSDGSF
FLYSKLTVDKSRWQQGNVFSCSVMHEALHNHYTQKSLSLSPG
C-IgG (Cetuximab modified with LC
C-terminal (G₄S)₃-LPETGS sortase A
recognition tag for conjugation):
Light chain
SEQ ID No. 8
DILLTQSPVILSVSPGERVSFSCRASQSIGTNIHWYQQRTNGSPR
LLIKYASESISGIPSRFSGSGSGTDFTLSINSVESEDIADYYCQQ
NNNWPTTFGAGTKLELKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGECGGGGSGGGGSG
GGGSLPETGS
Heavy chain
SEQ ID No. 9
QVQLKQSGPGLVQPSQSLSITCTVSGFSLTNYGVHWVRQSPGKGL
EWLGVIWSGGNTDYNTPFTSRLSINKDNSKSQVFFKMNSLQSNDT
AIYYCARALTYYDYEFAYWGQGTLVTVSAASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKRVEPKSCDKT
HTCPPCPAPELLGGPSVFLFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPGK
C-Fab (Cetuximab Fab fragment modified
with LC C-terminal (G₄S)₃-LPETGS
sortase A recognition tag for
conjugation and HC C-terminal
G4S-His6 tag for
purification):
Light chain
SEQ ID No. 10
DILLTQSPVILSVSPGERVSFSCRASQSIGTNIHWYQQRTNGSPR
LLIKYASESISGIPSRFSGSGSGTDFTLSINSVESEDIADYYCQQ
NNNWPTTFGAGTKLELKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEGGGGGGGGGSGG
GGSLPETGS
Heavy chain
SEQ ID No. 11
QVQLKQSGPGLVQPSQSLSITCTVSGFSLTNYGVHWVRQSPGKGL
EWLGVIWSGGNTDYNTPFTSRLSINKDNSKSQVFFKMNSLQSNDT
AIYYCARALTYYDYEFAYWGQGTLVTVSAASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKRVEPKSCDKT
HTGGGGSHHHHHH

### REFERENCES

[1] V. Boni, M. R. Sharma, A. Patnaik, Am. Soc. Clin. Oncol. Educ. B. 2020, e58-e74.
[2] Y. Y. Syed, Drugs 2020, 80, 1019-1025.
[3] A. Mullard, Nat. Rev. Drug Discov. 2020, 19, 659-659.
[4] P. Khongorzul, C. J. Ling, F. U. Khan, A. U. Ihsan, J. Zhang, Mol. Cancer Res. 2020, 18, 3-19.
[5] M. P. Deonarain, Drug Discov. Today Technol. 2018, 30, 47-53.
[6] B. T. Ruddle, R. Fleming, H. Wu, C. Gao, N. Dimasi, Chem Med Chem 2019, 14, 1185-1195.
[7] W. Liu, W. Zhao, X. Bai, S. Jin, Y. Li, C. Qiu, L. Pan, D. Ding, Y. Xu, Z. Zhou, S. Chen, Eur. J. Pharm. Sci. 2019, 134, 81-92.
[8] J. P. Higgins, A. Sarkar, E. T. Williams, A. Iberg, R. Waltzman, E. K. Willert, in Poster Sess. Abstr., American Association For Cancer Research, 2020, pp. P1-18-35.
[9] N. Aubrey, E. Allard-Vannier, C. Martin, F. Bryden, S. Letast, C. Colas, Z. Lakhrif, N. Collinet, I. Dimier-Poisson, I. Chourpa, M. C. Viaud-Massuard, N. Joubert, Bioconjug. Chem. 2018, 29, 3516-3521.
[10] Q. Li, A. Barrett, B. Vijayakrishnan, A. Tiberghien, R. Beard, K. W. Rickert, K. L. Allen, R. J. Christie, M. Marelli, J. Harper, P. Howard, H. Wu, W. F. Dall'Acqua, P. Tsui, C. Gao, M. J. Borrok, Bioconjug. Chem. 2019, 30, 1232-1243.
[11] L. Cao, Q. Li, Z. Tong, Y. Xing, K. Xu, J. Yijia Wang, W. Li, J. Zhao, L. Zhao, Z. Hong, Int. J. Pharm. 2020, 574, 118939.
[12] P. M. Glassman, L. R. Walsh, C. H. Villa, O. A. Marcos-Contreras, E. D. Hood, V. R. Muzykantov, C. F. Greineder, Bioconjug. Chem. 2020, 31, 1144-1155.
[13] I. Nessler, E. Khera, S. Vance, A. Kopp, Q. Qiu, T. A. Keating, A. O. Abu-Yousif, T. Sandal, J. Legg, L. Thompson, N. Goodwin, G. M. Thurber, Cancer Res. 2020, 80, 1268-1278.
[14] European Patent Application EP21171859.8
[15] E. Lobner, M. W. Traxlmayr, C. Obinger, C. Hasenhindl, Immunol. Rev. 2016, 270, 113-131.
[16] G. M. Thurber, M. M. Schmidt, K. D. Wittrup, Adv. Drug Deliv. Rev. 2008, 60, 1421-1434.
[17] G. Wozniak-Knopp, S. Bartl, A. Bauer, M. Mostageer, M. Woisetschläger, B. Antes, K. Ettl, M. Kainer, G. Weberhofer, S. Wiederkum, G. Himmler, G. C. Mudde, F. Rüker, Protein Eng. Des. Sel. 2010, 23, 289-297.
[18] K. M. Leung, S. Batey, R. Rowlands, S. J. Isaac, P. Jones, V. Drewett, J. Carvalho, M. Gaspar, S. Weller, M. Medcalf, M. M. Wydro, R. Pegram, G. C. Mudde, A. Bauer, K. Moulder, M. Woisetschläger, M. Tuna, J. S. Haurum, H. Sun, Mol. Ther. 2015, 23, 1722-1733.
[19] Z. Li, Y. Li, H. P. Chang, H. Y. Chang, L. Guo, D. K. Shah, Drug Metab. Dispos. 2019, 47, 1136-1145.
[20] M. Tuna, K.-M. Leung, H. Sun, M. Medcalf, S. Isaac, EGFR Binding Molecules, 2018, CA3030505A1.
[21] L. Baudino, Y. Shinohara, F. Nimmerjahn, J. Furukawa, M. Nakata, E. Martínez-Soria, F. Petry, J. V Ravetch, S. Nishimura, S. Izui, J. Immunol. 2008, 181, 6664-6669.
[22] R. L. Shields, A. K. Namenuk, K. Hong, Y. G. Meng, J. Rae, J. Briggs, D. Xie, J. Lai, A. Stadlen, B. Li, J. A. Fox, L. G. Presta, J. Biol. Chem. 2001, 276, 6591-6604.
[23] P. K. Mahalingaiah, R. Ciurlionis, K. R. Durbin, R. L. Yeager, B. K. Philip, B. Bawa, S. R. Mantena, B. P. Enright, M. J. Liguori, T. R. Van Vleet, Pharmacol. Ther. 2019, 200, 110-125.
[24] N. Nath, B. Godat, C. Zimprich, S. J. Dwight, C. Corona, M. McDougall, M. Urh, J. Immunol. Methods 2016, 431, 11-21.
[25] C. Cilliers, B. Menezes, I. Nessler, J. Linderman, G. M. Thurber, Cancer Res. 2018, 78, 758-768.
[26] E. Khera, C. Cilliers, S. Bhatnagar, G. M. Thurber, Mol. Syst. Des. Eng. 2018, 3, 73-88.
[27] S. Dickgiesser, M. Rieker, D. Mueller-Pompalla, C. Schröter, J. Tonillo, S. Warszawski, S. Raab-Westphal, S. Kühn, T. Knehans, D. Könning, J. Dotterweich, U. A. K. Betz, J. Anderl, S. Hecht, N. Rasche, Bioconjug. Chem. 2020, 31, 1070-1076.
[28] R. Gébleux, M. Briendl, U. Grawunder, R. R. Beerli, in Enzym. Ligation Methods (Eds.: T. Nuijens, M. Schmidt), Humana, New York, NY, 2019, pp. 1-13.
[29] I. Chen, B. M. Dorr, D. R. Liu, Proc. Natl. Acad. Sci. 2011, 108, 11399-11404.
[30] B. Shen, K. Xu, L. Liu, H. Raab, S. Bhakta, M. Kenrick, K. L. Parsons-Reponte, J. Tien, S. Yu, E. Mai, D. Li, J. Tibbitts, J. Baudys, O. M. Saad, S. J. Scales, P. J. McDonald, P. E. Hass, C. Eigenbrot, T. Nguyen, W. A. Solis, R. N. Fuji, K. M. Flagella, D. Patel, S. D. Spencer, L. A. Khawli, A. Ebens, W. L. Wong, R. Vandlen, S. Kaur, M. X. Sliwkowski, R. H. Scheller, P. Polakis, J. R. Junutula, Nat. Biotechnol. 2012, 30, 184-189.
[31] P. Strop, S. Liu, M. Dorywalska, K. Delaria, R. G. Dushin, T. Tran, W. Ho, S. Farias, M. G. Casas, Y. Abdiche, D. Zhou, R. Chandrasekaran, C. Samain, C. Loo, A. Rossi, M. Rickert, S. Krimm, T. Wong, S. M. Chin, J. Yu, J. Dilley, J. Chaparro-Riggers, G. F. Filzen, C. J. O'Donnell, F. Wang, J. S. Myers, J. Pons, D. L. Shelton, A. Rajpal, Chem. Biol. 2013, 20, 161-167.
[32] M. Dorywalska, P. Strop, J. A. Melton-Witt, A. Hasa-Moreno, S. E. Farias, M. Galindo Casas, K. Delaria, V. Lui, K. Poulsen, C. Loo, S. Krimm, G. Bolton, L. Moine, R. Dushin, T.-T. Tran, S.-H. Liu, M. Rickert, D. Foletti, D. L. Shelton, J. Pons, A. Rajpal, Bioconjug. Chem. 2015, 26, 650-659.
[33] M. Dorywalska, R. Dushin, L. Moine, S. E. Farias, D. Zhou, T. Navaratnam, V. Lui, A. Hasa-Moreno, M. G. Casas, T.-T. Tran, K. Delaria, S.-H. Liu, D. Foletti, C. J. O'Donnell, J. Pons, D. L. Shelton, A. Rajpal, P. Strop, Mol. Cancer Ther. 2016, 15, 958-970.
[34] M. Tuna, K.-M. Leung, H. Sun, M. Medcalf, S. Isaac, EGFR Binding Molecules, 2018, CA3030505A1.
[35] N. Nath, B. Godat, C. Zimprich, S. J. Dwight, C. Corona, M. McDougall, M. Urh, J. Immunol. Methods 2016, 431, 11-21.
[36] T. Riedl, E. Van Boxtel, M. Bosch, P. W. H. I. Parren, A. F. Gerritsen, J. Biomol. Screen. 2016, 21, 12-23.
[37] ThermoFisher Scientific, in Mol. Probes Handb., 2010, pp. 6-7.
[38] C. Wang, S. Chen, J. Caceres-Cortes, R. Y. C. Huang, A. A. Tymiak, Y. Zhang, J. Chromatogr. A 2016, 1455, 133-139.
[39] L. N. Tumey, F. Li, B. Rago, X. Han, F. Loganzo, S. Musto, E. I. Graziani, S. Puthenveetil, J. Casavant, K. Marquette, T. Clark, J. Bikker, E. M. Bennett, F. Barletta, N. Piche-Nicholas, A. Tam, C. J. O'Donnell, H. P. Gerber, L. Tchistiakova, AAPS J. 2017, 19, 1123-1135.

1. EGFR Fcab-drug conjugate or a pharmaceutically acceptable salt thereof, comprising the formula Fcab-(L)ₘ-(D)ₙ wherein:
a) Fcab comprises an EGFR Fcab,
b) L comprises a linker,
c) D comprises a drug,
d) m is an integer from 1-5 and n is an integer from 1-10.

2. EGFR Fcab-drug conjugate according to claim 1 wherein the EGFR Fcab is selected from the group consisting of: Fcab-1, Fcab-2, Fcab-3, Fcab-4, Fcab-5 and Fcab-6, having the amino acid sequences as set forth in SEQ ID Nos. 1-6.

3. EGFR Fcab-label conjugate comprising the formula Fcab-(L)ₘ-(La)ₙ wherein:
a) Fcab comprises an EGFR Fcab,
b) L comprises a linker,
c) La comprises a label,
d) m is an integer from 1-5 and n is an integer from 1-10.

4. Pharmaceutical preparation comprising at least one EGFR Fcab-drug conjugate according to claim 1.

5. Pharmaceutical preparation according to claim 4 comprising further excipients and/or adjuvants.

6. Pharmaceutical preparation comprising at least one EGFR Fcab-drug conjugate according to claim 1 and at least one further medicament active compound.

7. Process for the preparation of a pharmaceutical preparation, characterised in that an EGFR Fcab-drug conjugate according to claim 1 is brought into a suitable dosage form together with a solid, liquid or semi-liquid excipient or adjuvant.

8. Diagnostic composition comprising at least one EGFR Fcab-label conjugate according to claim 3.

9. A method for the treatment and/or prophylaxis of physiological and/or pathophysiological states, comprising administering to a subject in need thereof an effective amount of at least one EGFR Fcab-drug conjugate according to claim 1.

10. A method for the treatment and/or prophylaxis of a hyperproliferative disease or disorder, comprising administering to a subject in need thereof an effective amount of at least one EGFR Fcab-drug conjugate according to claim 1.

11. The method according to claim 10, wherein the hyperproliferative disease or disorder is cancer.

12. The method according to claim 11, wherein the cancer is an EGFR-positive cancer.

13. The method according to claim 11, wherein the cancer is selected from the group consisting of acute and chronic lymphocytic leukemia, acute granulocytic leukemia, adrenal cortex cancer, bladder cancer, brain cancer, breast cancer, cervical cancer, cervical hyperplasia, chorion cancer, chronic granulocytic leukemia, chronic lymphocytic leukemia, colon cancer, endometrial cancer, kidney cancer, biliary tract cancer, hepatoma, liver cancer, esophageal cancer, essential thrombocytosis, genitourinary carcinoma, glioma, glioblastoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, chondrosarcoma, multiple myeloma, mycosis fungoides, myeloid and lymphocytic leukemia, neuroblastoma, non-Hodgkin's lymphoma, non-small cell lung cancer, osteogenic sarcoma, ovarian carcinoma, pancreatic carcinoma, polycythemia vera, primary brain carcinoma, primary macroglobulinemia, prostatic cancer, renal cell cancer, rhabdomyosarcoma, skin cancer, small-cell lung cancer, soft-tissue sarcoma, squamous cell cancer, stomach cancer, testicular cancer, thyroid cancer and Wilms' tumor.

14. The method according to claim 11, wherein the cancer is selected from the group consisting of breast cancer, gastric cancer, stomach cancer, colorectal cancer, ovarian cancer, pancreatic cancer, endometrial cancer or non-small cell lung cancer

15. Set (kit) consisting of separate packs of
a) an effective amount of comprising at least one EGFR Fcab-drug conjugate according to claim 1, and
b) an effective amount of a further medicament active compound.
